(19) United States
(12) Patent Application Publication
(10) Pub. No.: US 2023/0173449 A1
(43) Pub. Date: Jun. 8, 2023

(54) SYSTEMS AND METHODS FOR MULTIPHASE DROPLET GENERATION FOR GENERATING SHAPED PARTICLES AND USES THEREOF

(71) Applicant: THE REGENTS OF THE UNIVERSITY OF CALIFORNIA, Oakland, CA (US)

(72) Inventors: Joseph De Rutte, Los Angeles, CA (US); Dino Di Carlo, Los Angeles, CA (US); Sohyung Lee, Los Angeles, CA (US)

(73) Assignee: THE REGENTS OF THE UNIVERSITY OF CALIFORNIA, Oakland, CA (US)

(21) Appl. No.: 17/996,927

(22) PCT Filed: Apr. 26, 2021
(86) PCT No.: PCT/US2021/029167

§ 371 Date: Oct. 22, 2022

Related U.S. Application Data
(60) Provisional application No. 63/018,346, filed on Apr. 30, 2020.

(51) Int. Cl. B01J 13/14; G01N 33/58

(52) U.S. Cl. CPC B01J 13/14; G01N 33/585

(57) ABSTRACT

A method of fabricating shaped particles is disclosed. The method involves generating a plurality of droplets within dispersion media (e.g., oil and surfactant), the plurality of droplets formed from a mixture of precursor materials that are in a miscible state. A stimulus or change of conditions is then introduced to the droplets so as to cause the mixture of precursor materials to become immiscible and phase-separate from one another. The phase-separated droplets are then crosslinked to form shaped particles. The stimulus or change of conditions may include one or more of the following: a change in temperature, a change in pH, a change in osmolarity, a change composition of the droplets, a change in the composition of the dispersion media. The shaped particles may be washed to remove un-crosslinked material and one or more affinity capture agents may be immobilized onto the shaped particles.

### RELATED APPLICATION

This application claims priority to U.S. Provisional Patent Application No. 63/018,346 filed on Apr. 30, 2020, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with Government support under grant number GM126414, awarded by the National Institutes of Health and under grant number N00014-16-1-2997, awarded by the U.S. Navy, Office of Naval Research. The Government has certain rights in the invention.

### TECHNICAL FIELD

The technical field generally relates to a method to fabricate small, sub-millimeter droplets composed of multiple phases. More specifically the technical field relates to approaches to generate multiphase droplets starting from an initial single miscible or semi-miscible phase. Droplets can then be used to fabricate shaped particles with unique morphologies and used for a number of applications.

### BACKGROUND

Shaped microparticles have been explored for numerous industrial uses, such as in biotechnology, medical devices, or in life science research. For example, spherical hydrogel microparticles can be assembled in vitro or in vivo to create porous scaffolds which allow rapid cellular ingrowth or act as a depot for delivering cells or drugs (see, e.g., U.S. Patent Application Publication No: US20170368224A1). Modulation of the shape of the particles to include completely enclosed voids or cavities opening to the surrounding environment can increase the effective space in assembled scaffolds, yielding improved cellular infiltration. Spherical particles with open cavities (e.g., particles with a crescent shaped cross-section) can also act as carriers for cell attachment, protecting cells from shear stress in the surrounding fluid. Similar crescent shape particles can also be used to isolate cells and scaffold the formation of uniform aqueous droplets in oil, or dropicles/particle-drops (see, e.g., International Patent Application Publication No. WO2020037214A1). These shaped particles can also be used to capture biomolecules released from attached cells, bind fluorescent molecules, magnetic beads, or other labels/barcodes to the captured biomolecules and may be analyzed using microscopy, flow cytometry, magnetic activated cell sorting, or other microfluidic single-cell systems.

Previous approaches to manufacture shaped microparticles have been limited in throughput. A number of approaches have been proposed to manufacture microscale materials with defined structures, such as cavities open to the surrounding fluid or completely enclosed voids. Photolithography and two-photon polymerization can generate asymmetric microparticles. Design of the molecular precursors enables polymerization following self-assembly to yield particles with a cavity and cubic particles. For example, Sacanna et al. produced colloids with a single spherical cavity through two step polymerizations of oil-in-water monomer droplets. See Sacanna et al., Lock and key colloids. Nature, 464 (7288), 575-578 (2010). However, previous manufacturing approaches, often dependent on very costly and specialized equipment, have not been scalable which is a significant barrier for the use of these materials in industrial applications.

Aqueous two-phase systems (ATPS), including a pre-polymer precursor, combined with flow focusing microfluidic devices have been used previously to manufacture crescent-shaped microparticles. See de Rutte et al., Massively parallel encapsulation of single cells with structured microparticles and secretion-based flow sorting, BioRxiv, https://doi.org/10.1101/2020.03.09.984245 (March, 2020). Flow focusing devices are used since co-flowing streams of the two aqueous phase precursors meeting at the droplet generation point allows quantities proportional to the flow rates of the two phases into each droplet generated. Mixing of the aqueous phases prior to introduction leads to local domains of the separate phases forming in the device prior to entering the droplet generator leading to non-uniform sizes for the crescent shaped void in the particles formed, including particles that may not contain cavities at all. Notably, a flow focusing design which has multiple co-flowing streams meeting prior to droplet generation is difficult to operate and balance all of the specific flow rates across numerous input channels.

### SUMMARY

In one embodiment, droplets are generated that contain miscible precursor phases that are then made into an immiscible state. Once in the immiscible state, the droplet is subject to one or more crosslinking operations to generate shaped particles. By starting with two precursor phases which are miscible, creating a homogenous solution, but then become immiscible upon a physical or chemical stimulus, the current invention overcome the challenges with scalable production in previous approaches. Induced phase-separation of droplets following microfluidic emulsification is a key to high-throughput production of monodisperse multiphased droplets (and shaped particles). A parallelized step emulsification device is used for droplet formation, which is compatible with a homogeneous solution, enabled scalable high-throughput generation of monodisperse homogeneous spherical droplets. By using triggered phase separation after droplet formation in a parallelized microfluidic step emulsification device or other high-throughput droplet generator it is possible to create uniform shaped particles with voids or cavities or completely enclosed voids. In the present invention, the transition between a single phase and multiple precursor rich and poor phases within a droplet can be induced by a number of methods, including, but not limited to: changing temperature, pH, osmolarity, pressure, concentration, molecular weight, or chemical composition.

The invention, according to one embodiment, generally includes the following components: two or more precursor materials are prepared or otherwise provided that are miscible, or have a long timescale for phase separation compared to the timescale for processing to form droplets. The mixed precursors are then formed into uniform sized droplets using microfluidic or other methods to create droplets. These may be uniform nanoliter scale or sub-nanoliter droplets (FIG. 3A). Once the droplets are formed, phase separation between the precursor materials is induced through physical or chemical stimulus (e.g., FIG. 3B using a temperature change as one example). Following phase separation, in some embodiments, one or more of the separated phases that was a polymer precursor is polymerized to form a shaped particle (e.g., FIGS. 1A, 1B, 3C). The shaped particles may include a family of shapes that include a spherical cavity subtracted from a spherical envelope, including crescent shapes, bowl shapes, moon shapes, capsule shapes, concentric sphere shapes and the like. The shaped particles may have a cavity open to the external environment or have a cavity surrounded by a material layer (e.g., enclosed cavity). The shaped particles can then be washed and isolated for use. The invention also includes methods of immobilizing affinity capture agents and cell adhesive regions onto the particles, and using the particles to isolate cells and/or biomolecules (e.g., FIG. 3D). Cells attached or otherwise adhered to the shaped particles may be flowed through, analyzed or sorted in flow cytometers, fluorescence activated cell sorters, and other single-cell analysis instruments. In some embodiments, the shaped particles can be used to template the formation of uniform water in oil emulsions, i.e., dropicles.

In one embodiment, a method of fabricating shaped particles includes the operations of: generating a plurality of droplets within dispersion media, the plurality of droplets formed from a mixture of precursor materials that are in a miscible state; introducing a stimulus or change of conditions to the plurality of droplets so as to cause the mixture of precursor materials to become immiscible and phase-separate from one another; and crosslinking one or more of the precursor materials in the phase-separated droplets to form shaped particles with a void or cavity. The shaped particles may then be washed to remove the un-crosslinked precursor materials to yield the final shaped particles. The stimulus or change of conditions may include one or more of the following: a change in temperature, a change in pH, a change in osmolarity, a change in the composition of the droplets, a change in the composition of the dispersion media.

In some embodiments, the mixture of precursor materials includes PEG-acrylate and gelatin and the shaped particle includes a void or cavity with the inner surface of the void or cavity having a localized cell adhesive region or layer formed thereon/therein. One or more affinity capture agents may be immobilized in or on the localized cell adhesive region or layer. The void or cavity and/or the localized cell adhesive region or layer may include cell adhesion moieties that aid in adhering cells to the shaped particle within the void or cavity. The cell adhesive region may include gelatin or fragments thereof, collagen or fragments thereof, hyaluronic acid or fragments thereof, poly-L-lysine, poly-D-lysine, other extracellular matrix proteins or fragments thereof, antibodies or fragments thereof with affinity to cell surface antigens, aptamers with affinity to cell surface antigens, oligonucleotides comprising complementary sequences to oligonucleotides present or conjugated to a cell surface, biotin, or streptavidin.

In another embodiment, a method of performing a cell secretion assay using shaped particles includes: (a) providing a plurality of shaped particles, each particle having a void or cavity formed therein; (b) loading cells into the voids or cavities of the plurality of shaped particles; (c) adding an affinity agent to the plurality of shaped particles specific to a cell secretion of interest; (d) incubating the plurality of shaped particles; (e) adding a stain, dye, label, barcode, or other secondary affinity capture agent specific to the secretion of interest on or in one or more of the plurality of shaped particles; (f) analyzing or sorting the plurality of shaped particles of operation (e) based on a signal formed or property generated by the stain, dye, label, barcode, or other secondary affinity capture agent specific to the cell secretion of interest on or in one or more of the plurality of shaped particles.

In another embodiment, a method of analyzing cells adhered to shaped particles with a flow cytometer includes: (a) providing a plurality of shaped particles, each shaped particle having a void or cavity formed therein, wherein the shaped particles are three-dimensional and includes one of: crescent shaped, bowl shaped, or moon shaped; (b) loading cells into the voids or cavities of the plurality of shaped particles; (c) flowing the plurality of shaped particles through a flow cytometer; and (e) analyzing the plurality of shaped particles of operation (c) based on a fluorescence and/or scatter signal measured with the flow cytometer.

In another embodiment, a shaped particle system includes a plurality of shaped particles, each shaped particle having a void or cavity formed therein, wherein the shaped particles are three-dimensional and are one of: crescent shaped, bowl shaped, or moon shaped, and wherein each shaped particle has a poly(ethylene glycol) (PEG) component located in a first region of the shaped particle and a cell adhesive component located in a second region of the shaped particle. In one embodiment, the cell adhesive component includes a localized gelatin region or layer that is disposed on the surface void or cavity or the shaped particles. The localized gelatin region or layer may include one or more affinity capture agents.

### BRIEF DESCRIPTION OF THE DRAWINGS

FIG. 1A illustrates one illustrative embodiment of a shaped particle. The shaped particle has a localized cell adhesive region or layer located at the inner surface of the void or cavity of the shaped particle. The localized cell adhesive region or layer may be functionalized with one or more cell adhesive moieties or capture agents specific to cell surface markers or biomolecules or secretions from a cell which is adhered to the shaped particle in the void or cavity.

FIG. 1B illustrates another illustrative embodiment of a shaped particle. This embodiment illustrates a spherical particle with hollow void or cavity therein. The hollow void or cavity is not open to the external environment but is surrounded by a shell of particle material.

FIG. 2 illustrates a flowchart of operations or steps used to generate the shaped particle and use the same for cell secretion capture/analysis.

FIGS. 3A-3D illustrate an overview of shaped particle fabrication using induced phase separation and their use. FIG. 3A illustrates polymer precursors containing a mixture of PEG-acrylate, gelatin, and photo-initiator are injected into a high-throughput microfluidic droplet generator to create a uniform two-phase water in oil emulsion. FIG. 3B schematically illustrates that by reducing the temperature of the emulsions, PEG and gelatin undergo phase separation to create a three-phase PEG/gelatin/oil system. Phase-separation of the droplets may be induced by a number of methods, including: changing temperature, pH, chemical composition, pressure and osmolarity. FIG. 3C illustrates the emulsion is exposed to UV light to selectively crosslink the PEG-rich phase and washed to recover the shaped particles with gelatin remaining localized on the cavity surface. FIG. 3D illustrates the shaped particles with localized surface chemistries act as cell carriers that protect cells from shear and show enhanced performance for single cell loading, secretion capture and live cell sorting using fluorescence activated cell sorters (as illustrated).

FIGS. 4A-4B illustrate the phase separation behavior of a solution of different concentrations of PEG-diacrylate (PEGDA) 1500 Da and fish gelatin depending on temperature. “X” in FIG. 4A indicates conditions in which a single phase is observed and “O” indicates conditions in which separate phases are observed. Two sets of conditions are highlighted in which a single phase of precursor materials transitions to separated phases upon a temperature reduction from 25° C. to 4° C. (i.e., X transitions to O). FIG. 4B shows images of phase separation of the various different combinations.

FIG. 5 illustrates the estimated binodal for solutions of gelatin and different molecular weight PEG as precursor materials at room temperature and 4° C. The binodal curves (lines) were estimated based on the experimental phase separation behavior of solutions for different concentrations of PEG and gelatin (points). The room temperature (RT) binodal curve is always offset to the top and right of the 4° C. binodal curve in the graphs.

FIGS. 6A-6H illustrate the fabrication of shaped particles using induced phase separation and the basis therefore. FIG. 6A shows the phase diagram of PEG and gelatin. The isothermal binodal curve is shown for 22° C. and 4° C. FIG. 6B illustrates that at concentrations between the binodal curves, phase separation can be induced by adjusting the temperature of the system. FIG. 6C shows example images of droplets at different gelatin compositions and temperatures (PEG concentration is 7.5% w/v). FIG. 6D illustrates the generation of unform single phase PEG/gelatin droplets using a highly-parallelized microfluidic droplet generator. FIG. 6E shows microscopy images of PEG/gelatin (6.3% w/v PEG and 4.5% w/v gelatin) droplets undergoing induced phase separation from a reduction in temperature and resulting in monodisperse shaped particles after UV polymerization. Green fluorescent images show the distribution of FITC conjugated gelatin during the process to aid in visualization. FIG. 6F illustrates the structure of the resulting particles can be modified by adjusting the composition of PEG and gelatin. Conditions are shown for crescent particles with different cavity ratios as well as fully enclosed hollow shell particles. Droplets are false colored to aid in visualization of PEG and gelatin phases. FIG. 6G illustrates the morphology of the crescent shaped particles. FIG. 6H illustrates confocal microscopy images of hollow shell particles to confirm morphology.

FIGS. 7A-7I illustrate shaped particles with gelatin coated cavities for cell loading and sorting. FIG. 7A includes confocal microscopy images showing the localization of gelatin on the inner cavity surface of the crescent shaped particles. FIG. 7B illustrates cell loading efficiency on shaped particles with different distributions of cell adhesive or binding moieties (No coating—PEG particles without RGD or gelatin, Uniform coating—PEG particles with RGD uniformly distributed, Localized coating—gelatin localized to the inner cavity). FIG. 7C illustrates how the fraction of shaped particles with single cells increases as the particle cavity size approaches the cell diameter. FIG. 7D illustrates the sorting of shaped particles loaded with cells based on CellTracker signal using FACS. FIG. 7E illustrates a graph of the viability of suspended cells (Cell) and cells loaded in gelatin-particle cavities after sorting. Cells bound to shaped particles showed significantly higher cell viability following sorting, suggesting these shaped particles provide protection from fluid shear stresses during the sorting process (****p<0.0001). FIG. 7F illustrates how cell-laden particles were sorted with high efficiency using FACS. FIG. 7G includes example images of live/dead stained cells after sorting that were either freely suspended or bound to shaped particles during the sorting. FIG. 7H is a graph of the viability of cells loaded on shaped particles which remained ˜80% over 5 days of culture for both non-sorted and sorted samples. FIG. 7I is a graph showing the average number of cells in shaped particles increases as they proliferated for both sorted and non-sorted samples.

FIGS. 8A-8C illustrate the spatial modification of particles with biomolecules using localized gelatin. FIG. 8A illustrates free amine groups on gelatin are conjugated with the affinity agent biotin using NETS-ester modification. FIG. 8B illustrates both biotin-modified PEG (Biotin-PEG) and biotin modified gelatin (Biotin-Gelatin) conjugated with AlexaFluor™ 488 conjugated streptavidin after fabrication. FIG. 8C illustrates fluorescence and confocal imaging showing increased fluorescence intensity in the inner cavity of Biotin-Gelatin shaped particles indicating localization of biotin.

FIGS. 9A-9H illustrate the localized conjugation of affinity capture agents reduces cross-talk for single cell secretion assays. FIG. 9A illustrates single cell secretion assays that were performed by first loading human IgG producing CHO cells into the shaped particles. After cell adhesion free biotin groups were conjugated with streptavidin and biotinylated antibodies against IgG. Cells were then incubated for different durations to accumulate secreted molecules on the surface of the shaped particles and captured secretions were then fluorescently labeled with fluorophore conjugated antibodies. FIG. 9B illustrates how the shaped particles were analyzed and sorted in high-throughput based on secretion signals using FACS. FIG. 9C illustrates a flow cytometry scatter plot of 0.5 hr incubated shaped particles, showing distinct scatter signal and distinguishable gates for shaped particles containing cells in comparison to empty shaped particles. FIGS. 9D and 9E include flow cytometry analysis and microscopy images that demonstrate that assays with Biotin-Gelatin shaped particles (FIG. 9E) led to higher secretion signal and lower background intensity on empty shaped particles as compared to Biotin-PEG shaped particles (FIG. 9D) due to the localized capture antibody in cavity. The dashed lines in the histograms show the threshold to exclude the bottom 99% of control empty shaped particles. FIG. 9F shows ROC analysis was performed to compare the classification accuracy for Biotin-PEG and Biotin-Gelatin shaped particle-based assays. FIG. 9G is a histogram of area under the ROC curve indicates that Biotin-Gelatin shaped particles enable more accurate classification than Biotin-PEG shaped particles due to reduced cross-talk (p<0.1) across all incubation times. FIG. 9H illustrates a histogram of particle fraction containing cells after incubating for 1 and 4 hours and sorting, reflecting the cross-talk differences between Biotin-PEG and Biotin-Gelatin.

FIGS. 10A-10C illustrate the characterization of the cell loading approach as compared to Poisson loading. FIG. 10A shows the shaped particles with uniform cell adhesive or binding moieties have loading statistics worse than the Poisson distribution, likely due to cell clustering and adhesion. Localized cell adhesive or binding moieties in shaped particles promotes deterministic cell loading and reduces the multiplet fraction below the number predicted by Poisson statistics. FIG. 10B shows that as the cavity size approached the average size of the cells, the fraction of shaped particles with single cells increased and multiple cells (multiplets) decreased. FIG. 10C shows the deterministic loading became more evident at higher cell seeding densities.

FIG. 11 illustrates a histogram showing cell viability percentage (%) for both non-sorted and sorted cells after a sorting operation was performed using a FACS device. The shaped particles protect the cells from hydrodynamic shear stress during the sorting process. Cells in suspension (Cell) had reduced viability compared to cells loaded in shaped particles (Particle).

FIGS. 12A and 12B illustrate the characterization of a secretion assay on the Biotin-Gelatin shaped particles compared to Biotin-PEG shaped particles. The graphs in FIG. 12A demonstrate that Biotin-Gelatin showed lower average background signal and higher secretion signal than Biotin-PEG over 4 hours. When a threshold of fluorescence intensity to have an only 1% false positive rate was defined, Biotin-Gelatin shaped particles had a higher fraction of shaped particles with positive signal above this threshold, indicating a significant reduction in cross-talk (FIG. 12B). For both shaped particles, the fraction dropped as the shaped particles were incubated longer than 2 hours.

FIG. 13 illustrates a schematic of a multi-channel step-emulsification microfluidic device which can be used for high-throughput production of droplets for induced phase separation-based manufacturing at high rates. In the top-down view the precursor solution is introduced into the left inlet and dispersion media (e.g., oil) is introduced into the right inlet. Droplets are formed as the precursor solution transit from the parallel microchannels to the taller reservoir region of the device. Droplets are collected in the downstream reservoir. Alternative embodiments like that of FIG. 3A use a common dispersion media channel and adjacent precursor solution channels.

FIGS. 14A and 14B schematically illustrate temperature-induced phase separation of droplets and an exemplary method to make phase-separated droplets using a temperature change. (FIG. 14A) Droplets with precursor materials that have temperature-sensitive miscibility such as PEG and gelatin can create two or more distinct phases as the temperature passes through the phase-separation point. (FIG. 14B) Example methods to induce phase-separation by changing the temperature (e.g., reduction in temperature). The temperature-controlled dispersion media flows into the emulsifier to achieve precise temperature control and phase-separation of the droplets before droplets exit the microfluidic device.

FIG. 15 illustrates a schematic to describe an orthogonal strategy to induce phase separation and shaped particle fabrication through step-wise polymerization reactions. First, partial polymerization is performed to increase molecular weight of one or more precursor materials, then phase separation is promoted by waiting a time period, and finally the phase separated droplets are fully crosslinked to generate particles based on the new phase-separated shape.

FIGS. 16A and 16B illustrate pH induced phase separation and particle crosslinking. (FIG. 16A) pH reactive PEG polymer, dithiol crosslinker, and dextran are mixed together in an acidic buffer (pH 5) below the binodal point and droplets are formed. Organic base is added through the oil phase to increase the pH to 7 and partially crosslink the PEG to effectively increase the molecular weight and induce phase separation. Additional organic base is added to increase the reaction rate and crosslink particles fully. Particles are then recovered by washing away the oil. (FIG. 16B) Example experiments showing fabrication of bowl-shaped particles using this approach. Fluorescent dye is conjugated to the PEG to help visualize PEG and dextran separation.

FIG. 17 illustrates phase-separation induced by evaporation of the precursor materials solvent in droplets. Evaporation of the solvent within a droplet can also be used to trigger phase separation of the precursor materials by leading to an effective increase in concentration of a precursor material.

FIGS. 18A-18B illustrates different internal shapes of phase-separated droplets are possible depending on the relative and overall concentration of the precursor materials. The effects of (FIG. 18A) precursor concentration, (FIG. 18B) ratio of precursor materials, and type of surfactants on the multi-phase droplet morphology.

FIG. 19 illustrates increasing the relative concentration of one precursor material in the precursor solution leads to different droplet morphologies after phase separation. These droplet morphologies can be transferred to different particle shapes following polymerization. In this schematic the A-rich phase contains a polymerizable precursor material and increase in relative concentration of the B-rich phase increases the relative resulting particle cavity volume.

FIG. 20 illustrates how adjusting the interfacial tensions between the different phases (A-rich, B-rich, oil) leads to different droplet morphologies. These droplet morphologies can be transferred to different particle shapes following polymerization. In this schematic the A-rich phase contains a polymerizable precursor material. In the depicted configuration the B-rich phase has a lower interfacial tension with oil. At low A-B interfacial tensions the B-rich phase can be fully enclosed resulting in enclosed hollow particles. At increased interfacial tensions between the A and B-rich phases, the B-rich phase will wet the oil interface more. The resulting particles have morphologies with increasing cavity opening diameters.

FIG. 21 illustrates polymerization of precursor materials in induced-phase separated droplets initiated by a change in pH. Organic base is introduced to droplets through the oil phase which is transported into droplets to increase the pH of droplets. The base removes a proton from a thiol group in the crosslinking agent, which initiates the crosslinking between precursor polymers and crosslinking agents.

FIG. 22 illustrates how particle morphology can be adjusted by adjusting both the time of phase separation as well as the polymerization rate, and a combination of the two. In one example, polymerizing the precursor material (A) prior to full phase separation can lead to particles with many small cavities (leftmost). Allowing for more phase separation can lead to particles with larger multiple cavities (middle left and right). Allowing for full phase separation leads to particles with a single main cavity that can be either enclosed or exposed to the surface of the particle.

FIG. 23 illustrates an overview of shaped particle based single cell secretion screening platform. (1) Prefabricated cavity-containing particles are seeded into a well plate and settle with their cavities remaining upright. Cells are seeded and adhere to the particle matrix through cell adhesive moieties, e.g., via integrin binding sites (RGD peptide). (2) Particles and associated cells are transferred into a tube and optionally are agitated by pipetting with biocompatible oil and surfactant to generate monodisperse compartments dictated by particle size (Dropicles). Alternatively, viscous additives can be added to the cell incubation media to reduce secreted molecule transport away from particles. (3) Cells are incubated to accumulate secretions on associated particles (Capture Sites) and transferred back to an aqueous phase for labelling. Particle/cells/secretions can then be analyzed and sorted using high-throughput commercial flow cytometers. Optionally, cell-carrier events can be gated and/or sorted based on the presence of a cell on the shaped particle, which gives a fluorescence or scatter signal above a threshold or within a gate.

FIGS. 24A-24F illustrate characterization of cell loading and dropicle formation. FIG. 24A illustrates shaped particles are loaded into wells and settle with their cavities mostly upright due to their asymmetric morphology. Cells are then seeded into the open cavities and adhere via integrin binding sites (RGD peptide) linked to the particle matrix (FIG. 24B). Fluorescence microscopy image of cells loaded into cavities where particles are labeled red and cell nuclei are labeled blue. Cell loading closely follows single-Poisson statistics (FIG. 24C) where λ is the average number of cells per particle. Loading fraction can be controlled by adjusting the seeding density. Error bars represent standard deviation across three separate samples. Fluorescent image of dropicles (FIG. 24D) formed by pipetting suspended particles with oil and surfactant. To aid in visualization shaped particles were labeled with a red fluorophore and a large molecular weight dye (blue) was included in the water phase. Droplet size analysis after emulsification shows two distinct subpopulations (FIG. 24E): smaller heterogeneous satellite drops and monodisperse dropicles (n=561). Nearly all droplets formed have either 0 or 1 shaped particle associated with them, with only a small fraction (<1%) containing 2 or more particles per droplet (FIG. 24F).

FIGS. 25A-25G illustrate the analysis of single-cell secretions using shaped particles. FIG. 25A illustrates an exemplary single-cell secretion assay workflow. Cells are first seeded onto shaped particles and washed. Biotinylated anti-human IgG Fc capture antibody is linked to the particle via biotin-streptavidin interactions. Cells are incubated on shaped particles to accumulate secretions, e.g., secreted antibodies. Optionally, dropicles are formed prior to the incubation step to reduce cross-talk and cells are incubated inside. After secretion accumulation, emulsions are broken if a dropicle-formation step is included. For each case, shaped particles 10 and associated cells 50 are then washed and stained using fluorescent secondary anti-human IgG secondary antibodies. FIG. 25B show example images of shaped particles with associated cells and secretion signal after performing a full secretion assay. Fluorescent images are overlaid onto a brightfield image. FIGS. 25C-25D illustrate the sweep of incubation times shows increased accumulation of secretions over time as expected, providing further validation of the assay. Signal intensity of only shaped particles with cells is shown. Mean intensity is consistent across triplicate samples (FIG. 25D). Fraction of cell population that is secreting human antibodies is seen in FIG. 25E. Secreting criteria defined as three (3) standard deviations above empty particle signal (dashed line shown in FIG. 25C). Significant signal over background is observed after as little as 1 hour of incubation. Secretion capture for shaped particles containing cells leads to higher signal intensity compared to shaped particles without cells when incubated in bulk without dropicle formation/compartmentalization in oil (FIG. 25F). Some cross-talk is observed when the secretion assay is performed on particles without the dropicle formation step, although this could be reduced by reducing the density of seeded shaped particles in the vessel or using other approaches as disclosed herein. Minimal cross-talk is observed when dropicle formation compartmentalizes cell secretions during the incubation step (FIG. 25G). Threshold in both (FIG. 25F) and (FIG. 25G) is set at three (3) standard deviations above the empty particle signal for the dropicle condition (FIG. 25G). Error bars represent s.d.

FIGS. 26A-26D illustrate sorting of cells based on secretions using commercial FACS. FIG. 26A schematically shows the selection of secreting cells of interest using FACS. Antibody (Ab) producing cells are mixed with non-producing cells (1:4 ratio), each stained with different colored Cell Tracker dyes and seeded on the shaped particles. A secretion capture assay is performed as previously described and samples are then collected, stained for secretions, and sorted using a commercial flow cytometer. FIG. 26B shows a microscopy image of samples after staining showing particles with Ab producing cells yield clear secretion signals which are not observed for non-producing cells. Samples are sorted by gating on the peak fluorescence intensity (FIG. 26B) of the secretion label channel. Collected samples are then imaged and analyzed. Spiked target cells were isolated across a range of initial fractions in a background of non-secreting cells (1:5-1:1000) and imaged to determine enrichment ratio (FIG. 26C) and purity (FIG. 26D).

FIGS. 27A-27C illustrate the selection of highly secreting cell subpopulations using FACS. The secreting population of cells were gated and sorted based on thresholds on IgG secretion signal and CellTracker (CT) Deep Red labelling (indicating the presence of a cell) (FIG. 27A). Both cells with any secretion signal disparate from background as well as the top 20% of secretors were separately sorted. Microscopy images before and after sorting show enrichment of secreting cells with fluorescent intensity proportional to the selection criteria (FIG. 27B). Isolated cells were expanded and bulk ELISA was performed to determine the production rate for the different subpopulations (FIG. 27C). Full experiment was performed with separate cell passages (n=4). Statistical significance based on standard two-tailed t-test (*p<0.05). Error bars represent s.d.

FIG. 28 illustrates ELISA measurements of each sorted sub-population of Anti-IL8 secreting CHO cells (FIGS. 27A-27C). For each sample (n=4) two separate sorting conditions were performed: (1) All cells with signal distinguishable from background (All Secretors) and (2) top 20% of secreting cells by measured secretion amount. An increase in IgG production was measured for all the sorted samples relative to the pre-sort control samples. In the highest producing sample (Top 20% Secretors, Sample D), a 58% increase in IgG production was measured relative to the pre-sort control sample.

FIGS. 29A-29E illustrate the characterization of cell viability and growth after dropicle formation and release. FIG. 29A shows brightfield and fluorescence microscopy image of cells encapsulated in dropicles. Biotinylated shaped particles are stained with Alexa Fluor 568 streptavidin and cells are stained with calcein AM. FIG. 29B shows that the cells maintained high viability after dropicle formation and release. Viability was assessed by staining with calcein AM and propidium iodide after recovering particles and cells from droplets. Live/dead assay was performed with a minimum of n=3 samples, cell number >1000 per condition. FIG. 29C illustrates that cells initially remain in the shaped particle cavities after releasing them. Colonies derived from single cells remain in the particle cavities during initial expansion. (FIG. 29D) After significant accumulation of cells in the cavities, cells begin to spread to the outside of the cavities and onto the well plate surface. Cell growth in shaped particles post dropicle release is comparable to cell growth on standard well plates (control) (n=3) (FIG. 29E). Growth is characterized by fold change in cell number over a 24 hr period as characterized by counting cell number using CellTracker staining.

FIGS. 30A-30B illustrate the identification of shaped particles containing cells using scatter signal with a flow cytometer. Here IgG producing cells and their secretions associated with shaped particles were labeled in different fluorescent channels and analyzed using On-Chip Sort (On-Chip Biotechnologies), a fluorescence activated cell sorter. The fluorescent channels were used to identify which events were (1) particles containing cells and which events were (2) particles with no cells (FIG. 30A). The resulting scatter plots of these populations were distinct indicating the two populations could be identified by scatter signal alone. The same sample was then analyzed starting from the scatter signal based on the gates determined in (A) the results of which are seen in FIG. 30B. (1) Gating scatter for events with cells resulted in 94.4% of events containing cells as determined by corresponding fluorescence signal. (2) Gating scatter for particle events without cells showed 98.9% purity as determined by corresponding fluorescence signal.

### DETAILED DESCRIPTION OF ILLUSTRATED EMBODIMENTS

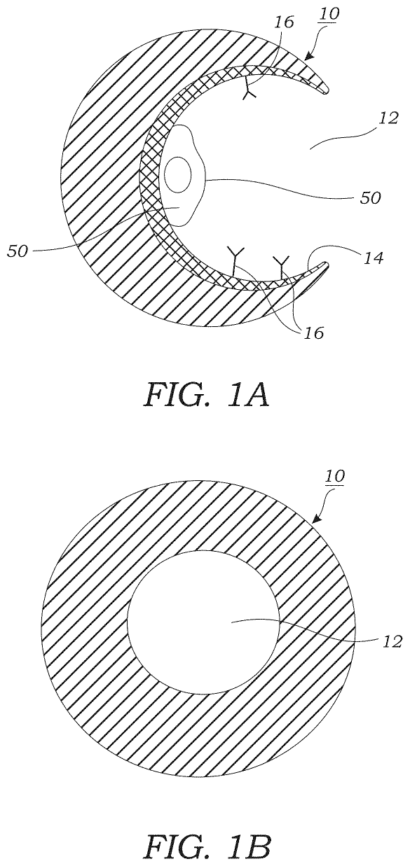

A method of fabricating shaped particles 10 is disclosed herein. FIGS. 1A and 1B illustrate two different illustrative shaped particles 10. The shaped particles 10 typically are micrometer sized particles (e.g., microparticles). Generally, the shaped particles 10 have a longest dimensional length of around 100 μm or less. For applications that require the loading of cells 50 into/onto the shaped particles 10, the shaped particles 10 typically have a minimum dimensional length of at least 10 μm. Of course, in other applications, there is no lower limit on the size of the shaped particles 10.

The shaped particle 10 includes a void or cavity 12. The void or cavity 12 may open to the external environment of the shaped particle 10 as illustrated in FIG. 1A. Alternatively, with reference to FIG. 1B, the void or cavity 12 may be completely enclosed by a surface of the shaped particle 10 (e.g., spherical particle with hollow void or cavity 12 therein). In still yet another embodiment, the shaped particle 10 may also include a particle (e.g., spherical particle) with a number of separate voids or cavities 12 distributed within the shaped particle 10 (e.g., FIG. 22). The shaped particles 10 may have a number of shapes including: crescent shaped, bowl shaped, moon shaped, capsule shaped, concentric sphere shaped. As explained herein, in some preferred embodiments, the shaped particles 10 preferably are designed to carry or hold cells 50 within the void or cavity 12.

As seen in FIG. 1A, in one embodiment, the shaped particles 10 have a localized cell adhesive region 14. The localized cell adhesive region 14 is preferably located along the inner surface of the void or cavity 12. In a preferred embodiment, the localized cell adhesive region 14 includes a cell adhesion component or material including, gelatin or a fragment thereof or collagen or a fragment thereof. The localized cell adhesive region 14 may be functionalized with biotin and/or streptavidin to enable binding directly to biotin or streptavidin labeled cells. Alternatively, or in addition, the localized cell adhesive region 14 comprises cell adhesive or binding moieties specific to other cell surface labels such as antibodies, aptamers, nucleic acids, oligonucleotides, and the like. The localized cell adhesive region 14 may be functionalized with one or more affinity capture agents 16. The affinity capture agent 16 may include, for example, biotin, streptavidin, a capture antibody, enzyme, protein, protein fragment, nucleic acid, aptamer, or the like. The affinity capture agent 16 may be secured to the localized cell adhesive region 14 using a linker molecule such as, for example, biotin and/or streptavidin. By populating the localized cell adhesive region 14 with affinity capture agents 16, the shaped particles 10 can be used to locally enrich secreted biomolecules or other products secreted or released from cells 50. In addition, the localized cell adhesive region 14 populated with the affinity capture agents 16 advantageously reduces unwanted leakage or crosstalk of secreted or released biomolecules interacting with other shaped particles 10. The localized cell adhesive region 14 populated with the affinity capture agent 16 also enables single cell secretion assays to be performed without the need for the formation of shaped particle 10 emulsions (i.e., no need for dropicle).

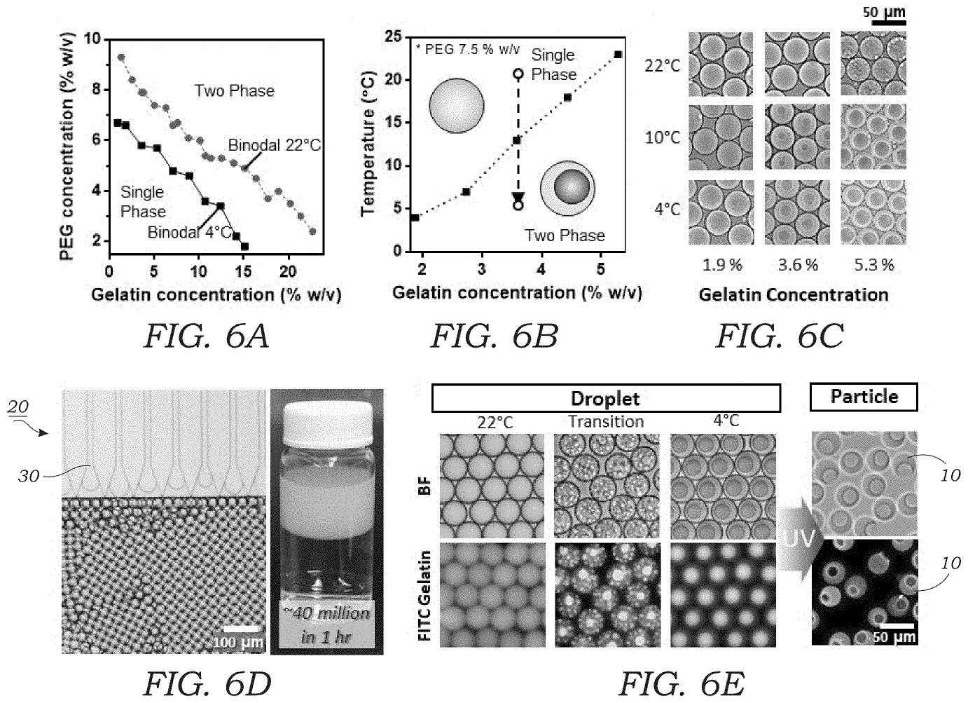

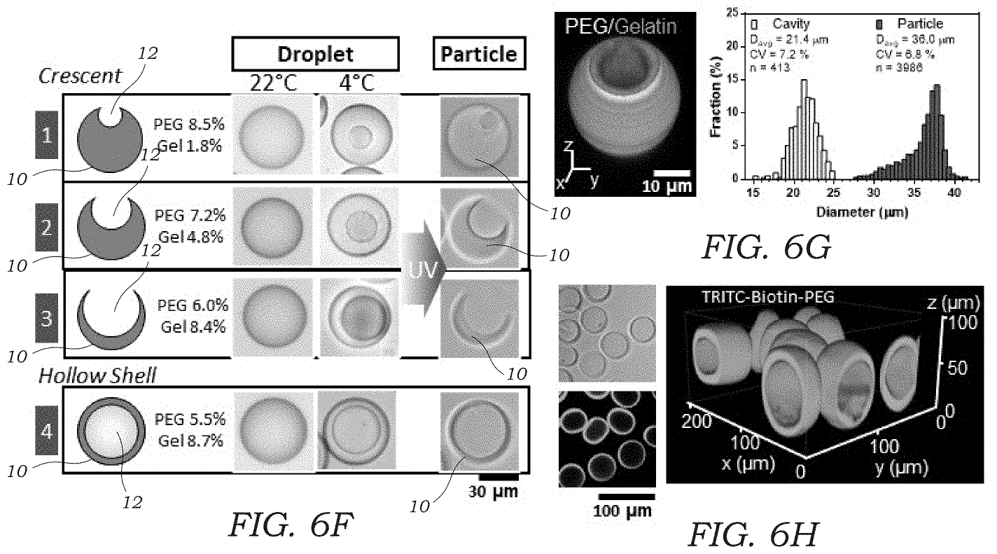

The localization of gelatin or a fragment thereof on the inner cavity surface of the crescent shaped particles 10 (i.e., localized cell adhesive region 14) is advantageous for cell microcarrier applications utilizing this shaped particle 10 geometry. It was found that during the shaped particle 10 manufacturing process gelatin molecules near the interface of the PEG-rich and gelatin-rich phases become trapped in the crosslinked surface. To this end, fluorescein isothiocyanate (FITC)-conjugated gelatin was used to visualize this localization effect using fluorescence and confocal microscopy (FIGS. 6E, 6G, 7A, 8C). It was found that even after vigorous washing steps that the gelatin or fragment thereof remained embedded in the surface of the shaped particle 10 indicating either physical entanglement and/or chemical bonding had occurred.

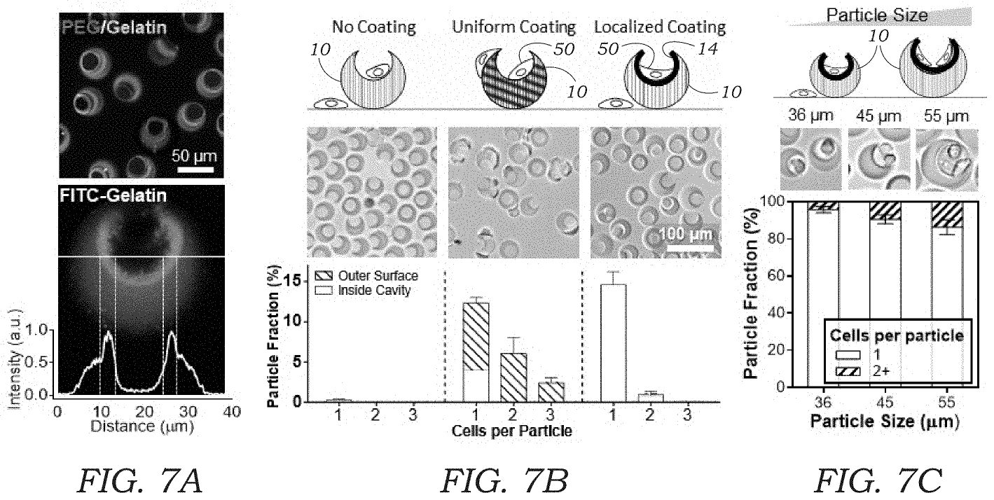

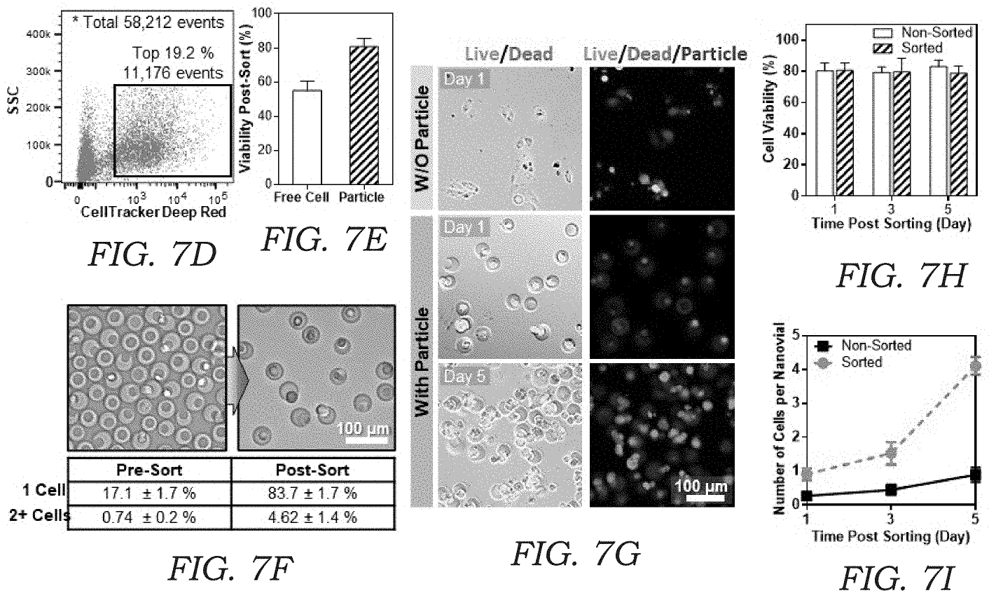

To investigate the effect of the localized cell adhesive region 14 (including gelatin as the cell adhesive component) on cell adhesion and growth in the shaped particles 10, the cell loading was compared on three different particles 10 with no binding motif, with uniform RGD moieties, and with a localized cell adhesive region 14, respectively (FIG. 7B). When Chinese Hamster Ovary (CHO) cells 50 were seeded at a 0.6:1 cell-to-particle ratio, the shaped particles 10 without cell adhesive or binding moieties had few cells 50 attached to the particles 10 (0.26±0.17% of particles 20 had adhered cells). More cells 50 were bound to RGD-coated shaped particles 10 as expected (20.73±1.77% of particles 10), but a significant fraction of cells 50 was bound to the outside of the shaped particle 10 (80.31% of cells 50 bound to particles 10). Shaped particles 10 with localized gelatin showed a significantly larger fraction of cells 50 bound to the particle void or cavity 12, 15.39±1.62% of particles, while less than 1.33±0.73% of them were bound to the outer surface (a 60-fold improvement as compared to uniformly coated particles 10). By avoiding external adhesion, the majority of cells 50 can be localized to void or cavity 12 which can help ensure loading of single cells 50 as well as reduce unwanted shear stress during handling steps, improving cell viability.

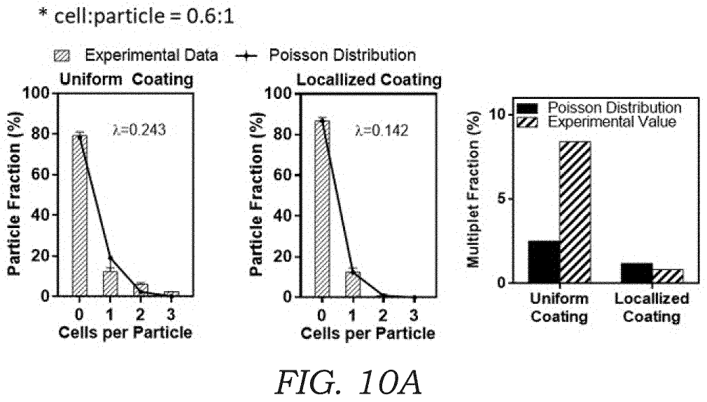

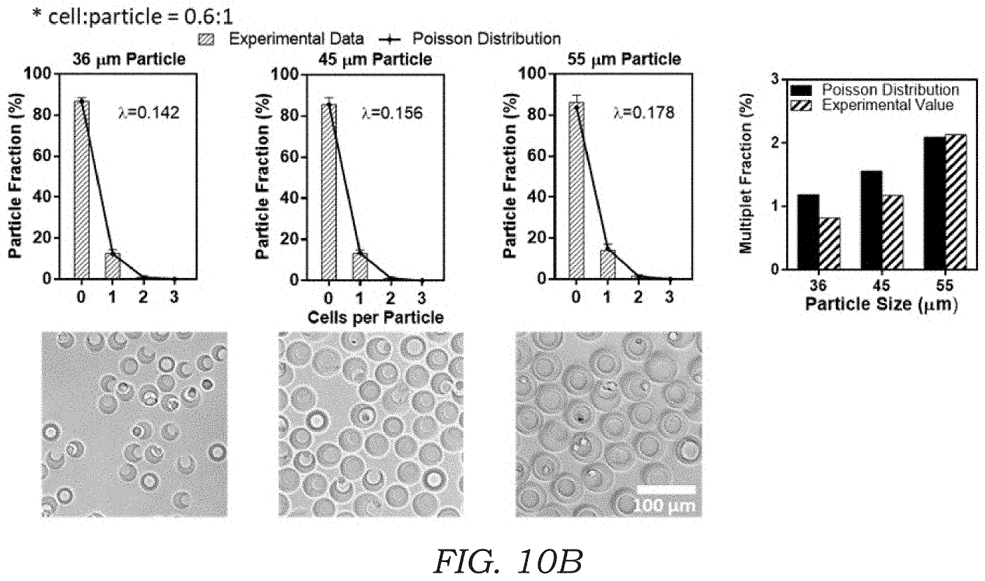

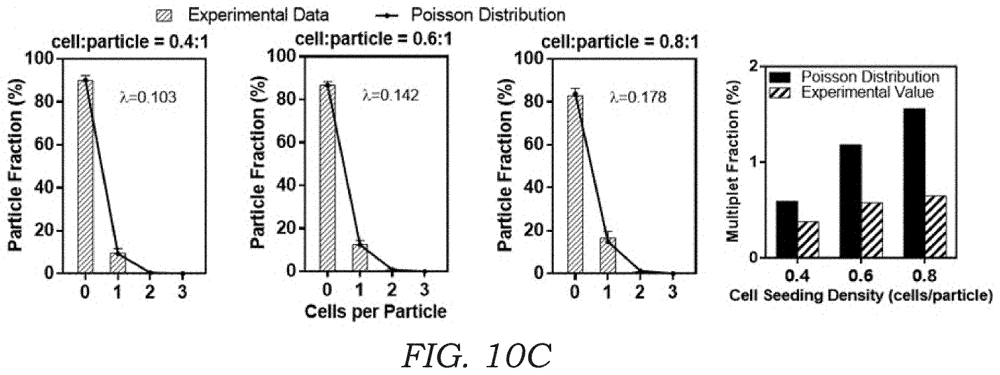

Localized adhesion combined with size exclusion effects of the void or cavity 12 enables deterministic loading of single cells 50 into the particle void or cavities 12. It was found that for the same cell seeding concentrations, shaped particles 10 with uniformly distributed adhesive or binding moieties yielded a significantly larger fraction of shaped particles 10 containing more than one cell 50 (>80% cell containing particles) than shaped particles 10 with gelatin localized to the void or cavity 12 (˜1% of cell containing particles). The high-multiplet fraction for the shaped particles 10 with uniformly distributed binding moieties was attributed to the larger fraction of cells 50 bound to the outside of the particles 10 and resulted in loading statistics slightly worse than Poisson loading. For shaped particles 10 with a localized cell adhesion region 14, the reduction in cells 50 binding to the outer surface combined with exclusion effects of the inner cavity size was found to improve loading of single cells 50 beyond distributions predicted by Poisson statistics. Both cells 50 loaded in RGD-coated shaped particles 10 and localized gelatin-shaped particles 10 showed high viability (>80% over 5 days of culture). Testing a range of shaped particle 10 sizes, it was found that as the void or cavity 12 approached the average size of the cells 50 (˜17 μm diameter) the fraction of shaped particles 10 with singlets increased and multiplets decreased (FIG. 7C and FIG. 10B). Preferably, the cell 50 characteristic dimension or diameter is >0.5 the cavity 12 characteristic dimension or diameter to obtain this beneficial effect. This effect became more evident at higher cell seeding densities where the fraction of multiplets was reduced by as much as 61% compared with Poisson loading (FIG. 10C). Although there is a tradeoff with loading efficiency for smaller void or cavity 12 sizes, as it is more difficult for cells 50 to fall into the cavity opening, it is expected that improved singlet loading can dramatically improve utility of the shaped particles 10 for applications where clonality is critical and overcomes one of the long-standing limitations of microfluidic platforms that are dictated by Poisson loading.

The shaped particles 10 with gelatin functionalized voids or cavities 12 facilitate cell growth and prevent cell death during standard assays that can induce high fluid dynamic shear stress such as fluorescence activated cell sorting (FACS). Both freely suspended cells 50 and cells 50 adhered in shaped particle voids or cavities 12 were sorted at high-throughput using FACS (˜270 events/second) (FIG. 7F) and cells 50 were expanded after sorting over several days (FIG. 7G). It was found that cells 50 bound in the shaped particle voids or cavities 12 showed significantly higher viability than unbound cells 50 after sorting (54.9% vs 80.0%, p<0.0001) (FIG. 7E and FIG. 11). It was found that there was no significant difference in viability of shaped particle-bound cells 50 that were sorted vs samples that were not sorted (FIG. 11). After sorting, the cells 50 loaded in shaped particles 10 proliferated well over five (5) days of culture and there were no significant differences in viability as compared to the non-sorted control group (FIG. 7H-7I). This improved viability is attributed to the void or cavity 12 acting as a protective shelter that reduces hydrodynamic shear stress on cells 50 during the sorting process. This improved viability is critical for improving the success of clonal expansion after sorting single cells 50 and could be advantageous for analysis and recovery of adherent cells 50.

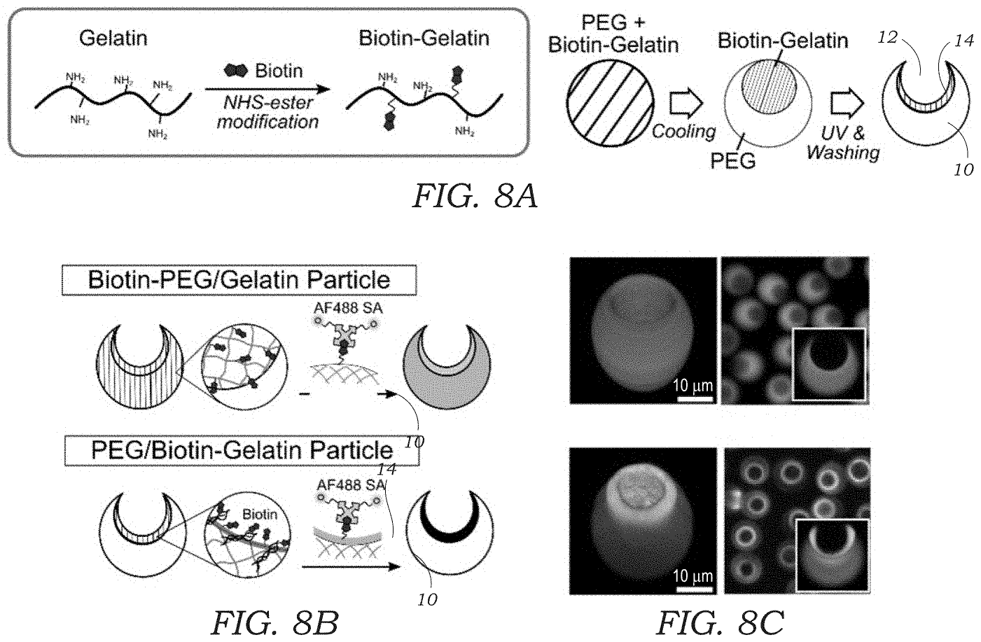

Gelatin localized on the inner particle surface enables facile spatial modification of the shaped particles 10 with other molecules of interest. Due to the abundance of functional handles such as free amines and carboxylic acid, gelatin is a convenient base for bioconjugation. For example, free amines can be easily linked to using N-hydroxysuccinimide (NHS) ester conjugates (FIG. 8A). Using an NHS-biotin conjugate, the inner void or cavity 12 of the shaped particles 10 is selectively modified with biotin, a biomolecule commonly used as a high affinity linker for antibodies, proteins, or oligonucleotides via the extremely high affinity biotin-streptavidin non-covalent interaction. In other embodiments, antibodies, proteins, or oligonucleotides can be directly conjugated to the inner void or cavity 12 of the shaped particles 10 using NHS esters, 1-ethyl-3-(3-dimethylaminopropyl) carbodiimide (EDC)/NHS, EDC/Sulfo-NHS or other bi-functional linkers used for bioconjugation known in the art. To visualize the difference in localization shaped particles 10 were fabricated with both biotin incorporated throughout the PEG backbone (Biotin-PEG) and biotin linked directly to the localized gelatin (Biotin-Gelatin) and stained with fluorescent streptavidin. Fluorescence and confocal microscopy revealed that the Biotin-PEG particles have a uniform distribution of biotin groups, while Biotin-Gelatin particles have a significant increase in fluorescence intensity around the inner surface of the cavity indicating a higher concentration of available biotin groups (FIGS. 8B-8C).

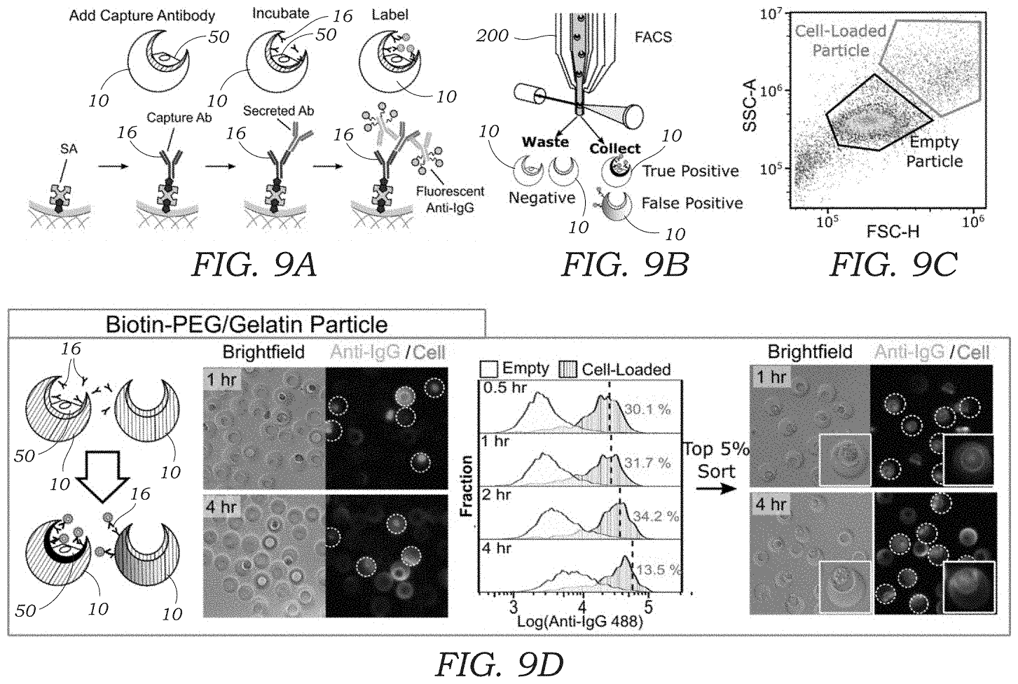

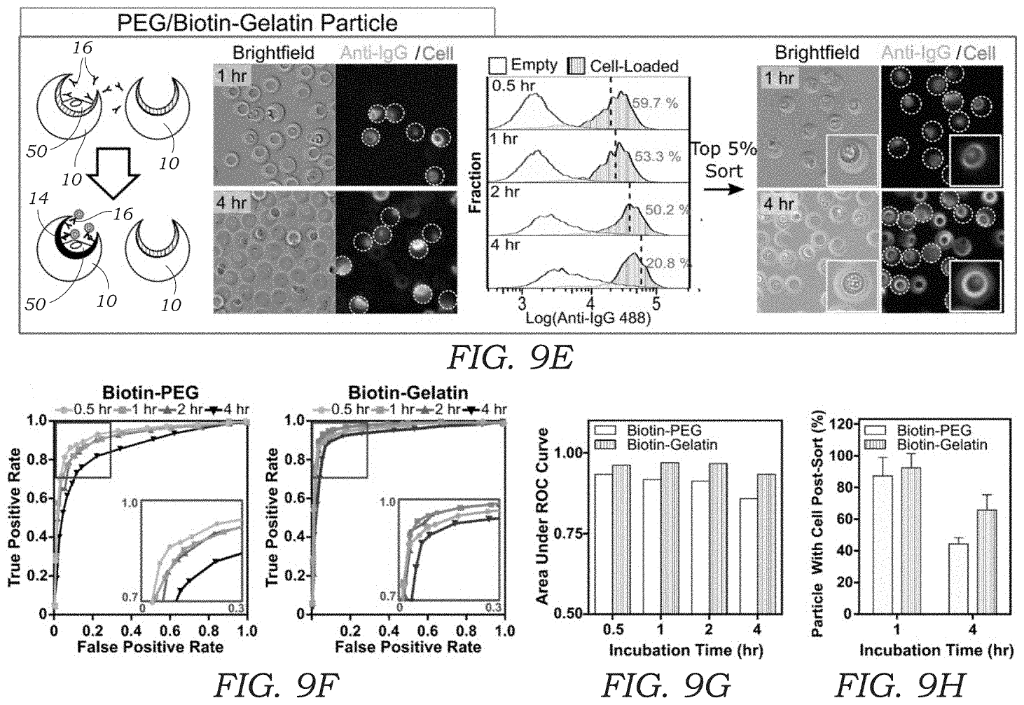

To characterize the effect of localized biotin on cross-talk, the secretion of a Human IgG against IL-8 produced by CHO cells 50 loaded on both Biotin-PEG shaped particles 10 and Biotin-Gelatin shaped particles 10 were measured without an emulsification step (FIG. 9A). The amount of cross-talk was characterized by introducing a population of empty control particles with a unique fluorescent label prior to the incubation step and the relative amount of secretions that were bound to these control “empty particles” were measured and compared to the cell containing population by flow cytometry (FIGS. 9B-9C).

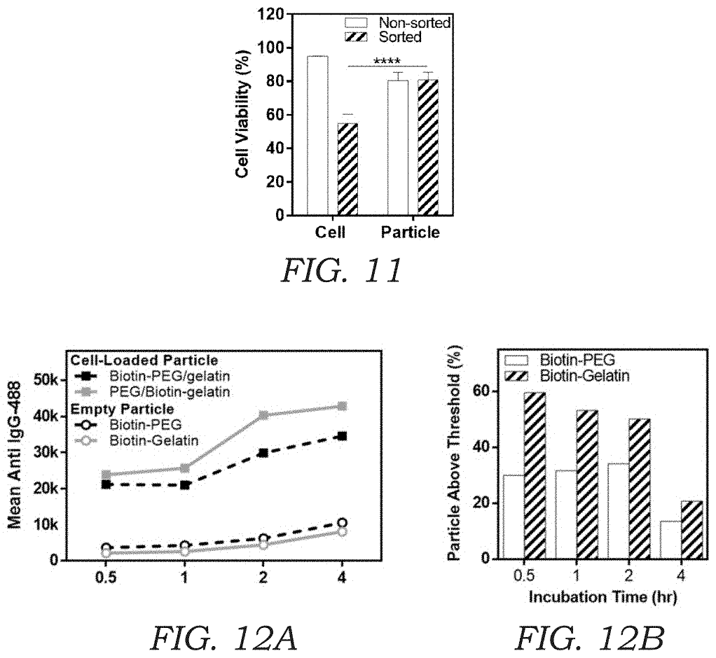

It was found that Biotin-Gelatin shaped particles 10 possessed a higher secretion signal and lower background intensity as compared to Biotin-PEG shaped particles 10, indicating that the localized capture antibody 16 in the void or cavity 12 of shaped particles 10 enriched the secretion signals and reduced secretion leak from cells to neighboring empty shaped particles 10 (FIG. 12A). A threshold of fluorescence intensity was defined to exclude the bottom 99% of control shaped particles 10 (1% false positive rate) and was used to identify the percent of cell-loaded shaped particles 10 (true positive rate) that were above this threshold (FIGS. 9D-9E). For Biotin-PEG shaped particles 10, it was found that ˜30-34% of cell-loaded shaped particles 10 possessed positive signal above this threshold when incubated within 2 hrs, which decreased substantially by 4 hrs to ˜13% due to an increase in crosstalk to control shaped particles 10 (FIG. 9D, FIG. 12B). For the Biotin-Gelatin shaped particles 10, ˜60% of cell-loaded shaped particles 10 were detectable above threshold after 30 minutes of incubation, which decreased slowly over time to ˜21% at 4 hrs, indicating a significant reduction in cross-talk as compared to Biotin-PEG shaped particles 10 (FIG. 9E, FIG. 12B).

To more fully characterize the capability of selecting out cell-containing shaped particles 10 from shaped particles 10 with signal cross-talk receiver operating characteristic (ROC) analysis was performed, a standard method to assess classification accuracy independent of a single threshold. For each condition, a curve of true positive rate versus its false positive specificity were obtained across a series of cutoff levels to depict the trade-off between the sensitivity and specificity (FIG. 9F) and the area under the curve (AUC) for each ROC curve was calculated (FIG. 9G). The ROC curve indicated that Biotin-Gelatin shaped particles 10 overall provide higher accuracy than Biotin-PEG shaped particles 10 with a minimum trade-off between true positive rate and false positive rate (FIG. 9F). While both shaped particles 10 allow accurate detection of secretions (AUC >0.85), Biotin-Gelatin shaped particles 10 showed significantly higher AUC values (AUC >0.93) than Biotin-PEG shaped particles 10 due to the reduced cross-talk (p<0.1) (FIG. 9G). This was also reflected in fluorescence microscopy images of the samples. The reduction in cross-talk for Biotin-Gelatin shaped particles 10 was further evidenced by inspecting shaped particles 10 sorted based on the top 5% of fluorescence intensity. For shorter incubation times (1 hr) both the Biotin-PEG and Biotin-Gelatin shaped particles 10 had a relatively low fractions of shaped particles 10 without attached cells: 7.1% and 12.1% respectively (FIGS. 9D, 9E, 9H). Note that these empty shaped particles 10 also likely include some amount of shaped particles 10 in which cells may be dislodged during processing and sorting. After 4 hrs the amount of empty shaped particles 10 sorted for the Biotin-PEG shaped particles 10 condition increased to 55.5±4.0% while Biotin-Gelatin shaped particle 10 sorts yielded only 33.8±8.6% empty shaped particles 10 (FIG. 9H). It was hypothesized that having the antibody binding sites 16 localized in the void or cavity 12 reduces the amount of leaked secretions reaching the binding sites of other particles 10 from convective transport. Convective flows into shaped particles 10 voids or cavities 12 are expected to be reduced given the boundary conditions, therefore requiring slower diffusive transport to reach the reactive surfaces in the voids or cavities 12. Further, it is possible that the higher concentration of binding sites 16 in the void or cavity 12 of the Biotin-Gelatin shaped particles 10 more efficiently captures secretions before they have time to diffuse out of the void or cavity 12 or be convected away, further reducing crosstalk. Taken together, locally functionalized shaped particles 10 can effectively capture the secretions from the single cells 50 with lower cross-talk, enabling more accurate analysis and sorting of desired sub-populations with a reduced number of processing steps then emulsification-based approaches.

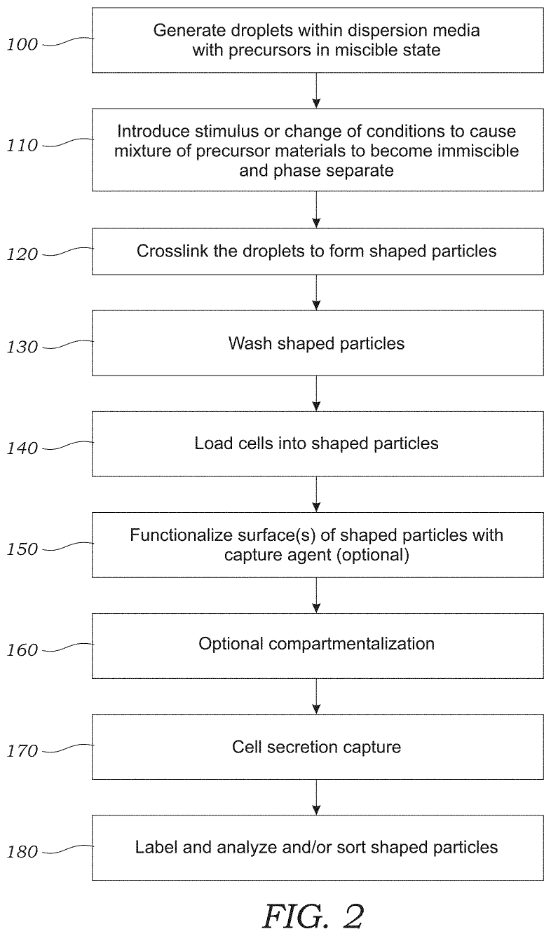

With reference to FIG. 2, in one embodiment, the method of fabricating shaped particles 10 involves the operations of: generating a plurality of droplets within dispersion media, the plurality of droplets including a mixture of precursor materials that are in a miscible state (operation 100 in FIG. 2). The dispersion media may include, for example, oil and a surfactant. The droplets may be aqueous-based and include the precursor materials and a crosslinking agent (e.g., photoinitiator). Next, in operation 110 of FIG. 2, a stimulus or change of conditions is introduced to the plurality of droplets so as to cause the mixture of precursor materials to become immiscible and phase-separate from one another. The stimulus or change of conditions may include one or more of the following: a change in temperature, a change in pH, a change in osmolarity, a change composition of the droplets, a change in the composition of the dispersion media. The phase-separated droplets are then subject to a crosslinking operation as seen in operation 120 to form the shaped particles 10. The shaped particles 10 may then be washed as seen in operation 130 to remove the non-crosslinked material to yield the final shaped particles 10.

The now-formed shaped particles 10 may then be used, for example, for the analysis of cells 50. As one example, the shaped particles 10 are used to capture and analyze molecules or secretions generated from cells 50. With reference to operation 140 of FIG. 2, cells 50 are loaded onto/into the shaped particles 10. Preferably, the cells 50 are loaded in the void or cavity 12 although in other embodiments the cells 50 may also be loaded onto the exterior surface of the shaped particles 10. Cell adhesive moieties, adhesion peptides (e.g., RGD peptide), extracellular matrix proteins or fragments thereof, antibodies, biotin/streptavidin, oligonucleotides and the like may be used to aid in binding cells 50 in the desired location(s) on the shaped particles 10. In one particular preferred embodiment, a single cell 50 is loaded into a single shaped particle 10. For example, adjusting the size of the void or cavity 12 to closely match the diameter of the cell 50 (e.g., a ratio of a characteristic dimension of the cell 50 to a characteristic dimension of the void or cavity 12 is within the range of about 0.5 to about 1) may help ensure single cell 50 loading within a single shaped particle 10.

The shaped particles 10 with the loaded cells 50 may optionally be subject to a functionalization operation 150 to functionalize the surface(s) of the shaped particles 10 with one or more affinity capture agents 16. For example, after loading the shaped particles 10 with cells 50, the shaped particles 10 may be washed and, in one preferred embodiment, the inner surface of the void or cavity 12 and/or the localized cell adhesive region 14 is functionalized with one or more affinity capture agents 16 that are specific for biomolecules or other secretion products from the cells 50. Alternatively, the material that forms the inner surface of the void or cavity 12 and/or the localized cell adhesive region 14 may have been pre-functionalized with the one or more affinity capture agents 16. The shaped particles 10 then undergo an optional compartmentalization operation 160 as seen in FIG. 2. In this operation, the shaped particles 10 and associated cells 50 are transferred into a tube, vessel, or container and subject to an agitation operation with biocompatible oil and surfactant to generate monodisperse aqueous compartments surrounded by an oil phase. Of course, it should be appreciated that the presence of the localized cell adhesive region 14 populated with the one or more affinity capture agents 16 enables secretion assays to be performed without the added complication of forming emulsions. This improves the overall workflow and integrates more easily with downstream (e.g., FACS) analysis and sorting as there is no need for the formation of emulsions.

As seen in operation 170, the biomolecules or secretions from the cells 50 in the shaped particles 10 are captured with affinity capture agents 16 located on or within the shaped particles 10. In one embodiment, the affinity capture agent 16 includes one or more capture antibodies or fragments thereof that are used to capture the biomolecules or secretions. In one particular embodiment, the affinity capture agent 16 is localized to the surface of the void or cavity 12 and/or the localized cell adhesive region 14 of the shaped particle 10. For example, the localized cell adhesive region 14 functionalized with capture antibodies as the affinity capture agent 16 can be used to locally enrich secreted products from captured cells 50. In some embodiments, the captured biomolecules or secretions can then be labelled (operation 180 in FIG. 2) using, for example, a fluorophore conjugated antibody which can be used for analysis and/or sorting of the shaped particles 10 (and cells 50 contained therein). For example, a fluorescent activated cell sorter (FACS) may be used to analyze and sort shaped particles 10 containing the adhered cells 50. It should be appreciated that the invention is not limited to fluorophore conjugated antibodies, as other labels may be used including: a stain, dye, magnetic particle, oligonucleotide, enzyme, metal isotope tag, and/or or other secondary affinity capture agent specific to the secretion of interest on or in one or more of the plurality of shaped particles 10. Depending on the label used a corresponding analysis and/or sorting approach may be used in operation 180, including microscopy, magnetic activated cell sorting (MACS), single-cell sequencing, mass cytometry, and other microfluidic analysis approaches.

Precursor Materials

In a preferred embodiment the precursor materials include two components of an aqueous two-phase system, wherein under an initial condition (e.g., temperature, pH, osmolarity, concentration) the precursor materials are miscible (e.g., they do not phase separate) or have a long time constant for phase separation. The time constant for phase separation should generally be greater than the time required for droplet formation and downstream processing. Typical time constants may be greater than 1 hour for phase separation in a centimeter scale diameter vessel with 1 mL of precursor of materials (the time constant is much longer in larger volumes). More preferred time constants are greater than 2 hours or, to have improved uniformity of the distribution of the ratio of precursor materials in each droplet formed, greater than 5 hours. In some embodiments shorter time scales, less than one hour and greater than 1 min, may be used if the precursors are thoroughly mixed in flow prior to droplet generation. For sub-millimeter-scale droplet volumes, phase separation typically takes place from seconds to minutes once phase separation is induced. The precursor materials should undergo phase separation to form at least one precursor rich and precursor poor volume under a change in conditions or in response to an applied stimulus (e.g., temperature, pH, osmolarity, concentration). Materials with these features include hydrophilic polymeric materials, charged polymeric materials, polysaccharides, salts, or polymeric materials which undergo gelation reactions. Preferably, precursor materials include components of an aqueous two-phase system (APTS).

In one exemplary embodiment, the precursor materials include a poly(ethylene glycol) (PEG) component and a gelatin component (e.g., for temperature-mediated phase separation within the droplets). The poly(ethylene glycol) component in some embodiments includes a multi-arm poly(ethylene glycol) with reactive groups (i.e., greater than or equal to two (2) reactive groups). Example reactive groups include, vinylsulfone, acrylate, maleimide, norbornene, methacrylate, acrylamide, methylsulfone, thiol, amine, or a mixture of two or more of the above. In some embodiments 4-arm PEG-vinylsulfone, 4-arm PEG-acrylate, PEG-diacrylate, 4-arm PEG-maleimide, 4-arm PEG-norbornene, 4-arm PEG-methacrylate, 4-arm PEG-acrylamide, 4-arm PEG-thiol, PEG-dithiol, 4-arm PEG-amine, or a mixture of two or more of the above are used. The PEG component can have various molecular weights, for example 700 Da, 1,500 Da, or 10,000 Da, although other molecular weights between 500-40,000 Da may also be used.

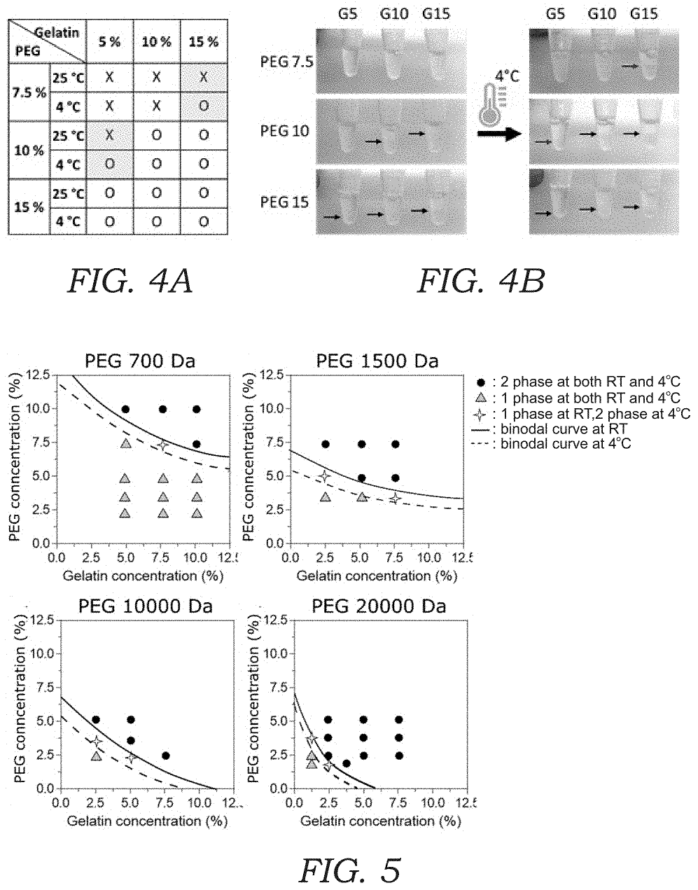

The gelatin component includes a mixture of denatured collagen. The gelatin is derived from bones or skins of animals such as pigs, cows, sheep, chickens, fish bones, fish skins, fish scales, or a combination of thereof. In some embodiments, gelatin includes gelatin extracted from cold-water fish (Sigma, Product #G7765-1L). For example, gelatin derived from fish still remains liquid at 4° C., aiding in flow unlike for porcine-derived gelatin. In some embodiments a gelatin component includes gelatin modified with reactive groups such as gelatin methacryloyl (GelMA), allyl modified gelatin (e.g., pentenoyl gelatin), alkyne functionalized gelatin, thiol-modified gelatin, biotin-modified gelatin, and fluorophore-modified gelatin. The weight fraction of the PEG component and gelatin component in water as solvent are tuned to achieve miscibility at room temperature, but phase separate at lower temperature (e.g., 0-4° C.) (FIGS. 4A and 4B). In some embodiments the weight fraction of PEG varies between 5% and 15% and the weight fraction of gelatin varies between 5% and 15%. In preferred embodiments, 700 Da PEG has a weight fraction of 15% and gelatin has a weight fraction of 15%, 1,500 Da PEG has a weight fraction of 7.5% and gelatin has a weight fraction of 15%, 1,500 Da PEG has a weight fraction of 10% and gelatin has a weight fraction of 5%, 10,000 Da PEG has a weight fraction of 7.5% and gelatin has a weight fraction of 5%, 10,000 Da PEG has a weight fraction of 5% and gelatin has a weight fraction of 10%. Generally, preferred conditions will have weight percentages that occur between the two lines in the phase diagrams in FIG. 5 and FIG. 6A.

In embodiments where a droplet of precursor materials is used to form a crosslinked shaped particle 10, the precursor materials further includes one or more crosslinking agents. In one embodiment, the crosslinking agent includes one or more photoinitiators. Exemplary photoinitiators include, but are not limited to, dithiothreitol (DTT); benzoin methyl ether; benzoin isopropyl ether; 2,2-diethoxyacetophenone (Irgacure™ 651 photoinitiator); 2,2-dimethoxy-2-phenyl-1-phenylethanone (Esacure™ KB-1 photoinitiator); dimethoxyhydroxyacetophenone; 2-methyl-2-hydroxy propiophenone; 2-naphthalene-sulfonyl chloride; 1-phenyl-1,2-propanedione-2-(O-ethoxy-carbonyl)oxime; 2,4-diethyl thioxanthone; 2-tert-butyl thioxanthone; 2-chlorothioxanthone; 2-propoxy thioxanthone; 2-benzyl dimethylamino-1-(4-morpholinophenyl)butan-1-one (Iracure 369™ photoinitiator); 2-methyl-1-[4-(methylthio)phenyl]-2-morpholino propan-2-one (Iracure 907™ photoinitiator); 2-hydroxy-4′-(2-hydroxyethoxy)-2-methylpropiophenone (Irgacure-2959™ photoinitiator); lithium phenyl-2,4,6-trimethylbenzoylphosphinate (LAP); triethanolamine; N-vinyl caprolactam; benzophenone; benzil dimethyl ketal; diethoxyacetophenone; dibutoxyacetophenone; methyl phenyl glycoxylate; 2-ethylthioxanthone; 2-isopropylthioxanthone; phenyl 2-hydroxy-2-propyl ketone; 4-isopropylphenyl 2-hydroxy-2-propyl ketone; 4-n-dodecylphenyl 2-hydroxy-2propyl ketone; 4-(2-hydroxyethoxy)phenyl 2-hydroxy-2propyl ketone; 4-(2-acryloyloxyethoxy)phenyl 2-hydroxy-2-propyl ketone; 1-benzoylcyclohexanol; and Eosin Y.

Uniform Droplet Generation

In a preferred embodiment, monodisperse droplets formed with precursor materials are generated using an emulsification process by which a precursor solution, including a mixture of precursor materials in a homogeneous or mixed state (i.e., prior to significant phase separation), is partitioned into microscale droplets suspended in dispersion media. The precursor solution is a liquid including materials that are mentioned herein in the Precursor Materials description. The dispersion media is a fluid that is immiscible with the precursor solution and prevents the coalescence of generated droplets. The dispersion media includes an oil which can include oils and organic solvents that are immiscible with the precursor solution materials, such as mineral oil, fluorinated oils (Novec™, HFE 7500, Fluorinert oil FC40), silicone oils, or other oils known in the art to support the formation of stable microdrops. The emulsification method may use microfluidic approaches, vortex mixing, homogenization, membrane emulsification, dispensing processes, spray and electrohydrodynamic spray. Internal obstructions in a flow device may also be used to cause droplet formation to occur. For instance, baffles, ridges, posts, or the like may be used to disrupt liquid flow in a manner that causes the fluid to coalesce into fluid droplets. The microfluidic approaches include use of microfluidic devices that induce the precursor solution to form individual droplets (e.g., co-flow, flow-focusing, T-junction, and step emulsification devices). Preferably, the emulsification method creates substantially monodisperse droplets (e.g., with a coefficient of variation in diameter of less <10% and more preferably with a coefficient of variation <5%).

The method of using an induced phase separation is advantageous in that it also enables more simplified microfluidic device geometries. For example, devices with a single input/inlet each for precursor solution (inlet 28) and dispersion media (inlet 34). The method can also enable higher throughput production of droplets, for example in being compatible with step emulsification devices 20 as described herein which require only a single input of precursor solution and single dispersion media. In some cases where the generated droplets are not monodispersed, the droplets of the desired size, or polymerized particles, may be separated by one or more filtration processes. For example, tangential flow filtration, inertial microfluidic based separation, or other filtration methods for sub-millimeter particles known in the art. For example, these filtration processes can reduce the CV in formed particle characteristic diameters from >10-15% to CVs <5% in final particles isolated.

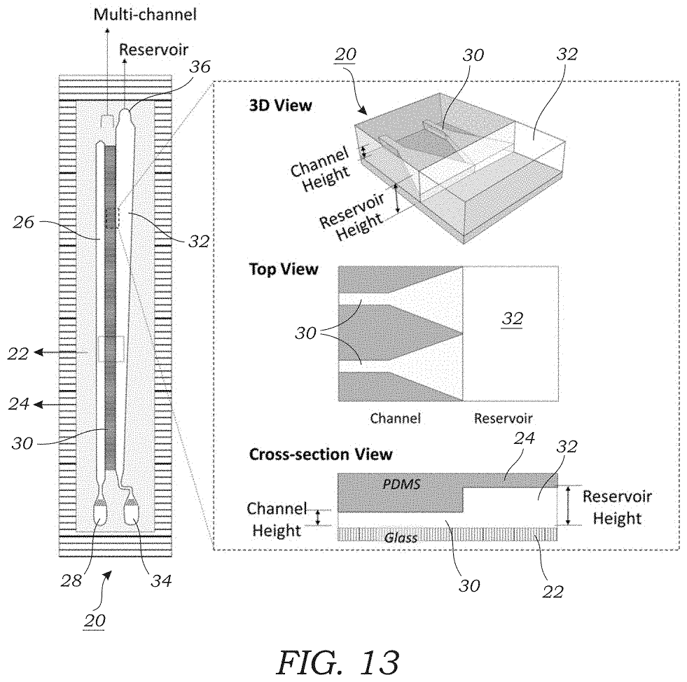

In one exemplary embodiment, an aqueous precursor solution, including 7.5% w/w PEG 1500 Da and 15% w/w fish gelatin, and a dispersion media, Novec™™ 7500 oil with 0.5% Pico-Surf™, are separately injected into a step-emulsification microfluidic device 20 (FIG. 13) to generate monodisperse water-in-oil droplets. The flow rates of dispersion media and precursor solution are 40 μL/min and 10 μL/min respectively. In the step-emulsification microfluidic device 20, a glass substrate 22 is bonded or secured to a PDMS top 24 that defines a first channel 26 coupled to an inlet 28 that receives the precursor solution. The height of the first channel 26 may vary, for example, between about 5 to about 500 μm. The first channel 26 includes a plurality of side channels 30 that communicate with a second channel 32 that acts as a reservoir channel for the formed droplets and has a larger height than the height of the side channels 30. The plurality of side channels 30 have a height that is smaller than the height of the second channel 32. This forms the steps in the step-emulsification device 20. For example, the height of the side channels 30 may range from about 5 to about 500 μm. The height of the second channel 32 (e.g., reservoir channel) may range from about 100 to about 500 μm. In some embodiments, the height of the side channels 30 is the same as the height as the first channel 26, although this is not necessary. There may be hundreds to thousands of these side channels 30 which operate in parallel to generate droplets. The second channel 32 includes its own inlet 34 that receives the dispersion media and an outlet 36 for removal of the droplets. The size of droplets that are generated varies depending on the geometry of the device 20. For example, a step-emulsification microfluidic device 20 with 25 micrometer channel heights (for side channels 30) produces ≈90 μm diameter droplets while a device with an 11-micrometer channel height (for side channels 30) produces ≈50 μm diameter droplets.

Inducing Phase Separation

The phase-separation of droplets can be induced through a change in temperature, pH, osmolarity, composition of the droplets or the dispersion media or a combination thereof.

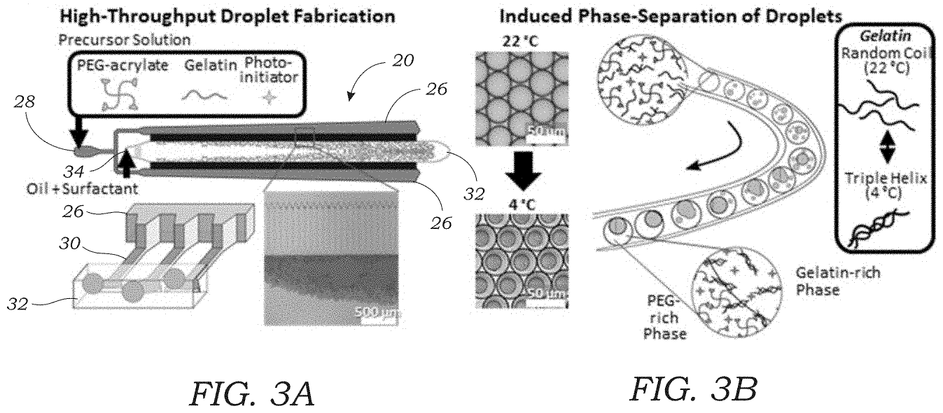

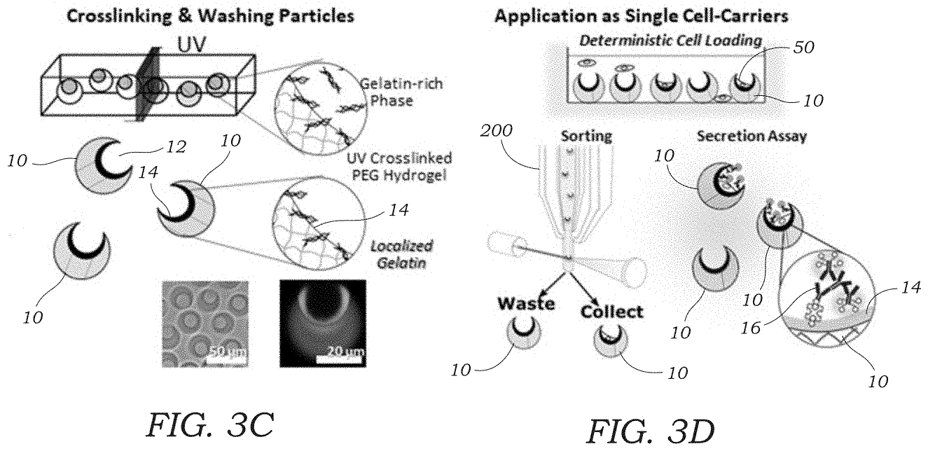

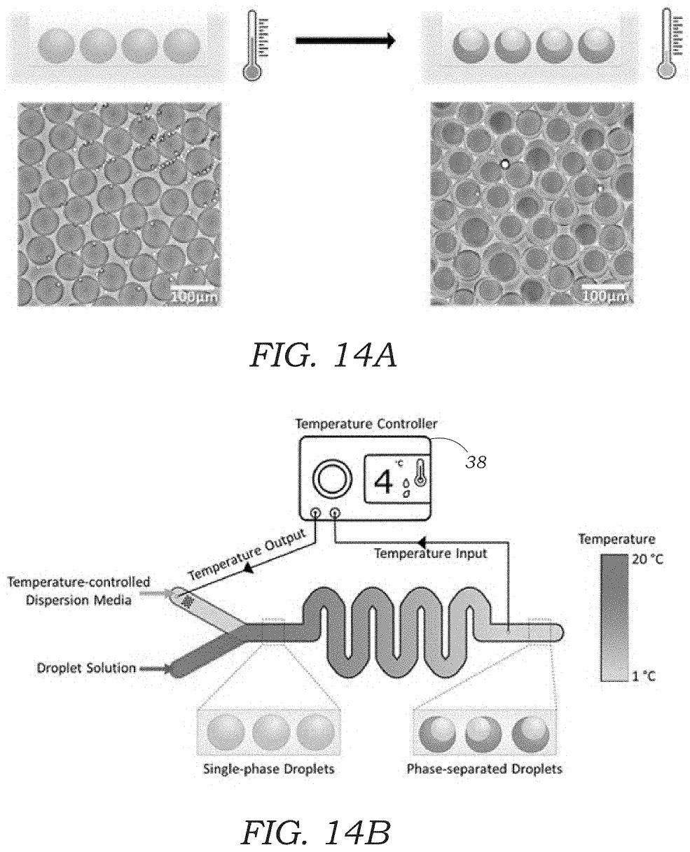

In a preferred embodiment, droplets including precursor materials that have temperature-sensitive miscibility create two or more distinct phases due to a change in temperature (FIGS. 3B, 14A). In one exemplary embodiment, to induce phase-separation of two or more precursor materials within droplets, the temperature of droplets can be changed by incubation of the droplets in an appliance, instrument, or compartment which is cooled or heated (e.g., refrigerator, oven, or the like). For example, droplets containing aqueous solution of 7.5% w/w PEG and 15% w/w gelatin in water are emulsified in Novec™™ 7500 fluorinated oil with 0.5% v/v Pico-Surf™ surfactant at 20° C. The water-in-oil droplets are moved to a 4° C. refrigerator and incubated for 2 hours to phase-separate into a PEG-rich phase and a gelatin-rich phase with a crescent shape (e.g., FIGS. 1A, 6F). In another exemplary embodiment, a temperature controller 38 (e.g., Peltier cooler, cooling fluid line) can be placed adjacent to or in thermal contact with the surface of the microfluidic device 20 where the droplet generation occurs. Alternatively, the dispersion media, in which temperature is set by a temperature controller 38 in an upstream container, is flowed into the emulsifier to achieve precise temperature control and phase-separation of the droplets before droplets exit the microfluidic device (FIG. 14B). In another embodiment, the outlet tubing containing the water-in-oil drops can be submerged in a temperature controlled liquid bath (e.g., water, oil, coolant) to control temperature after droplet production. Various other heat exchangers may be used to heat or cool the droplets as needed.

Temperature-based induced phase separation can be achieved in general with other systems in which one or more of the precursor materials have a phase change that is temperature dependent. In some embodiments this occurs when one or more precursor materials possess a temperature dependent gelation mechanism (e.g., gelatin, agarose, collagen, etc.). Gelation leads to an effective increase in the molecular weight of the precursor material which may shift the location of the binodal curves for phase separation when forming a mixed precursor solution (FIGS. 5, 6A, 6B). Other example precursor material combinations include PEG/Dextran, PEG/Agarose, Gelatin/Dextran, PNIPAM/Alginate, PNIPAM/PAAM, Pluronic/Dextran.

One specific example of a temperature-based phase separation utilizes the phase separation of PEG and gelatin derived from fish. Phase separation of PEG and gelatin is dependent on both the concentration of each component and the temperature of the system. At concentrations above the binodal curves, the system undergoes phase separation to create PEG-rich and gelatin-rich regions within microscale water in oil droplets. For concentrations below the binodal curves, PEG and gelatin were miscible. The binodal boundary was found to be lowered by decreasing the temperature, which was attributed to favored interactions between gelatin molecules at lower temperatures (FIGS. 6B-6C). By using compositions of the PEG/gelatin solutions located at points between the 4 and 22° C. binodal curves (FIG. 6A) a transition is enabled from a miscible solution to a phase-separated state induced by the temperature change. This was confirmed for both bulk solutions and in droplets (FIG. 6C).

The droplets are generated in the step-emulsification microfluidic device 20 described herein starting at a single-phase composition between the binodal lines (FIG. 6D), and then inducing phase separation by reducing temperature to create uniform multiphase geometries. As the temperature of the system is reduced very small domains of the gelatin-rich phase form within the larger drops, which coalesce and coarsen over time to form a single large spherical gelatin-rich domain in each drop (FIG. 6E). The phase-separated droplets were then exposed to UV light to crosslink the PEG portion, yielding shaped particles 10 with crescent-shaped cross-sections having an average diameter of 36.0 μm (CV=6.8%) and average void or cavity diameter of 21.4 μm (CV=7.2%) (FIG. 6G) after washing steps. Shaped particles 10 were generated at rates of 40 million/hour which is ˜8 times faster than previous methods that used a single flow-focusing device. Shaped particles 10 of various sizes can be generated by using step-emulsifier devices 20 with different channel heights. The morphology of the ATPS droplets and resulting shaped particles 10 can be adjusted by changing the composition of PEG and gelatin. The compositions affect both the relative volumes of the PEG-rich and gelatin-rich regions as well as the balance of interfacial tensions between the PEG-rich, gelatin-rich, and oil phases. Compositions were found that resulted in repeatable fabrication of crescent shaped particles 10 with exposed voids or cavities 12. Increasing the concentration ratio of gelatin to PEG resulted in droplets with a higher volume fraction of the gelatin-rich phase, and shaped particles 10 with a larger exposed cavity when crosslinked (FIG. 6F). Compositions were also found that result in shaped particles 10 with completely enclosed cavities (i.e., hollow shell particles 10) (FIG. 1B and FIG. 6H). For hollow shell shaped particles 10 the hydrogel matrix used comprises pores that allow transport of species with molecular weight <˜100 kD through the shell in one embodiment, although hydrogel matrix porosity can be tuned to further adjust this molecular weight cutoff.

In one embodiment, a change in pH affects the electric charge of precursor materials and leads to phase-separation. Phase-separation occurs when mixing entropy is too low to compensate for the positive mixing enthalpy. When one of the precursor materials is a polyelectrolyte, the charged polymer and its counter ions that ensure electroneutrality should be restricted in one phase in order to phase-separate, causing an interfacial electric potential difference. Therefore, as the charge on one of the polymers is increased, it increases entropic cost for phase separation and the critical point of mixing is expected to shift to higher concentrations. For example, when a polyelectrolyte, blended with other precursor material, is in a pH far from its isoelectric point, the polymer is highly charged and maintains homogeneity due to the high entropic cost. However, as the pH nears its isoelectric point, the mixing entropy decreases and leads to phase-separation. In one exemplary embodiment, droplets containing aqueous solution of 7.5% w/w PEG 1500 Da and 20% w/w fish gelatin with isoelectric point 6, are emulsified in Novec™™ 7500 fluorinated oil with 0.5% v/v Pico-Surf™ surfactant in pH 3. After fabrication of droplets, the pH of droplet is adjusted to pH 6-7 by adding organic bases such as triethylamine (TEA) through the dispersion media. Phase-separation due to a pH change can be achieved with other systems in which one or more of precursor materials are polyelectrolytes whose miscibility is highly dependent on pH. Other example precursor material combinations include Gelatin/Dextran, Gelatin/PEG, Dextran/PEG and Agarose/PEG.

In another embodiment, phase separation can be induced by adjusting the salinity of the droplet phase. For example, some polymer-polymer ATPS systems or polymer-salt systems are sensitive to salt concentration or salt type. By changing the concentration of dissolved salt, phase separation can be induced. Effective salt concentration can be adjusted by a number of ways. For example, dehydrated salt can be added through the oil phase. In another embodiment, a salt with solubility dependent on pH can be added in precipitant form prior to droplet formation. After droplet formation the pH can be adjusted (e.g., through addition of organic acids or bases through the oil phase) to dissolve the salt precipitant and adjust salinity. For example, calcium carbonate precipitates or nanoparticles can be added to the precursor material at neutral or slightly basic pH. Acetic acid can then be added to the oil to decrease the pH and dissolve the calcium carbonate.

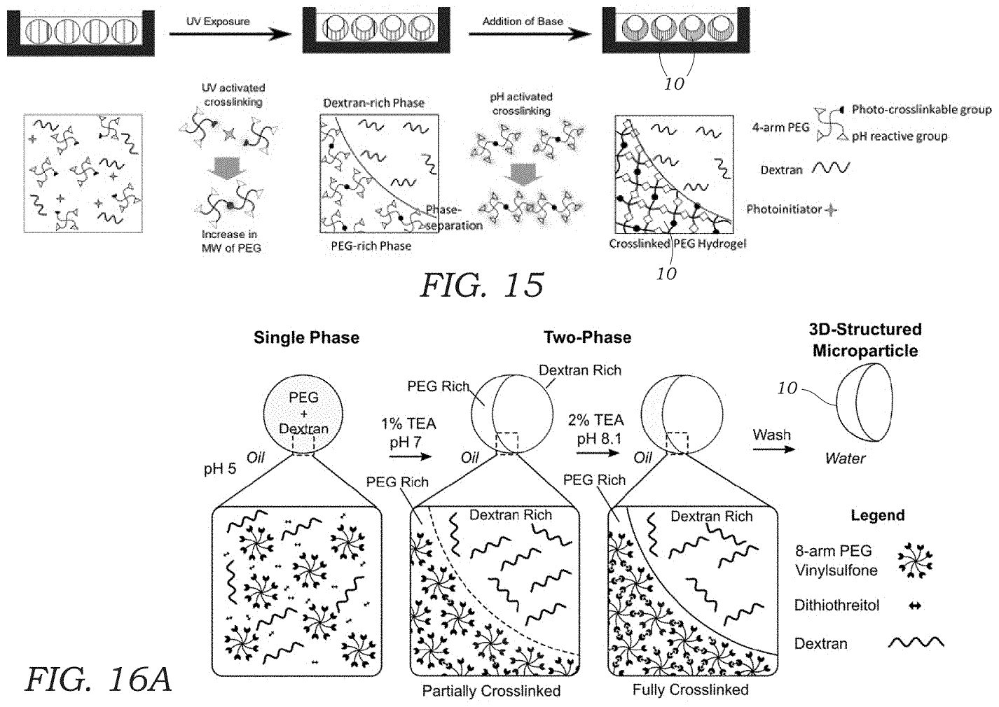

In addition to induced phase separation due to a temperature change and a pH change, other stimuli can be introduced through the surrounding dispersion media to induce phase separation. Phase separation of polymer-polymer ATPS systems as well as polymer-salt ATPS systems is sensitive to the molecular weight of the polymers. Polymerization, initiated through a number of approaches such as a temperature change, pH change in the dispersion media, or photoactivation of radical initiators from exposure to light can lead to a change in molecular weight of one or more precursor materials that leads to phase separation as the binodal shifts. For example, pH of the surrounding oil dispersion media can be modulated with triethylamine to initiate polymerization after droplet formation. In some embodiments, the pH is modulated to an intermediate value to initiate slow polymerization reactions and then the pH is modulated to another extreme value to lead to rapid crosslinking for particle manufacture at rates one to two orders of magnitude higher than the intermediate pH value. pH may be shifted to be higher or lower depending on the nature of the polymerization reaction. In embodiments when crosslinked particles are manufactured, the polymerization reaction that induces phase separation can be maintained to more fully crosslink particles. Alternatively, the polymerization reaction used to induce phase separation can be separate or orthogonal from the polymerization reaction used for crosslinking particles. Functional groups can be categorized by the types of stimuli to be polymerized, such as UV exposure, a certain pH or temperature range or increase in salt concentration. Functional groups from more than two different categories should be involved in the overall reaction to have two different steps of polymerization. As described in FIG. 15, in one embodiment, a dextran and a 4-arm PEG that has an arm containing a photo crosslinkable group and three pH-dependent crosslinking groups is used as one of the initial precursor materials in a solution at an initial pH from <6 to ˜7-7.5. To assist crosslinking of each group, DTT and a photoinitiator should be included in the precursor solution. Once droplets are formed, a photo-polymerization reaction occurs leading to phase separation over a 15 min to 2-hour period. Once phase separation has occurred the pH is increased to >8 in order to crosslink particles fully. pH changes can be achieved for example, through use of organic base in the oil phase, such as triethylamine. In some embodiments two different functional groups may be present on different precursor materials. For example, PEG in the precursor solution may have 10 mol % PEG-VS (a pH reactive group) and 90 mol % of PEG-Norbornene (a photo-crosslinkable group). PEG-VS, having a smaller fraction of total PEG in the precursor solution can be polymerized first by an increase in pH to induce phase-separation but not create a complete polymer network, then PEG-Norb can be later crosslinked upon exposure to light in the presence of a photoinitiator to fully crosslink particles 10.

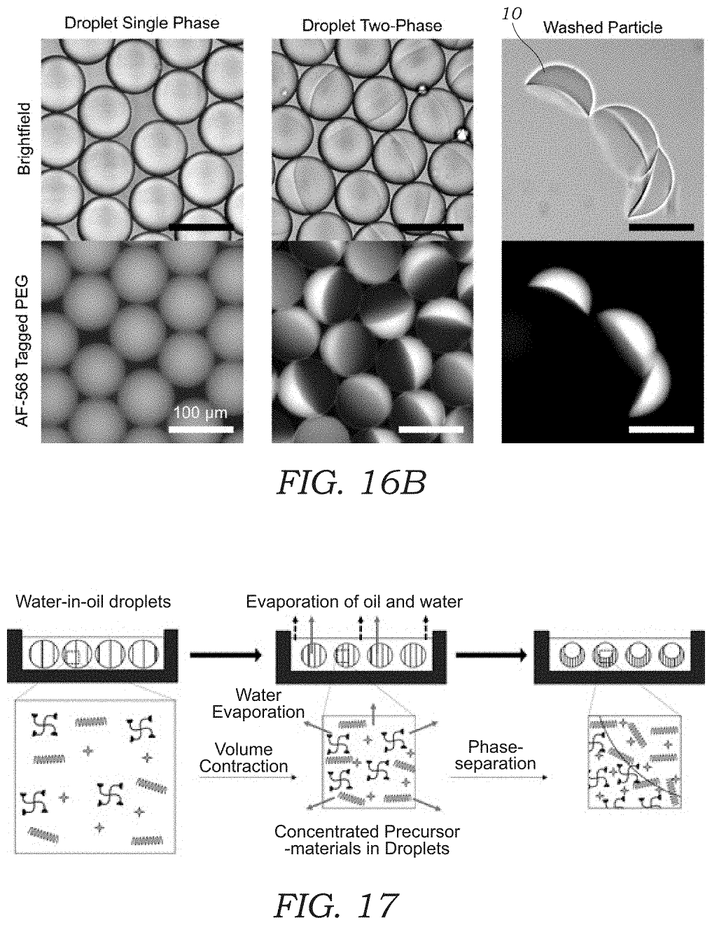

In one exemplary embodiment, pH mediated partial crosslinking is used to induce phase separation between a pH reactive multi-arm PEG and dextran (FIGS. 16A and 16B). 2% w/w 8-arm PEG vinylsulfone, 3.2 mM dithiotreitol, and 6.6% w/w 40 kDa dextran are mixed together in 0.3 M triethanolamine buffer at slightly acidic pH (e.g., pH 5). At this condition precursors were found to be sufficiently miscible. The precursor is then emulsified into Novec™™ 7500 fluorinated oil with 0.5% v/v Pico-Surf™. Additional Novec™™ 7500 fluorinated oil containing 1% triethylamine by volume is then added to the oil phase of the emulsion at equal volume to the precursor phase to increase the pH to ˜7. The emulsion is then gently agitated and let sit for 2 minutes to partially crosslink the PEG polymer and induce phase separation. Additional Novec™™ 7500 fluorinated oil containing 2% triethylamine by volume is then added to the oil phase of the emulsion at equal volume to the precursor phase to increase the pH again to ˜8.1. The further increase in pH accelerates crosslinking to preserve the particle morphology. The crosslinked shaped particle 10 can then be transferred into aqueous phase through a series of washing steps as mentioned in the paragraph beginning with: Fabrication of crescent-shaped particles using temperature induced phase separation.

Evaporation of the solvent within a droplet can also be used to trigger phase separation of the precursor materials by leading to an effective increase in concentration which occupies a new location on the phase diagram (FIG. 17). Evaporation can be achieved through the oil phase over time at room temperature (20° C.), preferably a 24 hour to 48-hour time period. Evaporation can be accelerated above room temperature, for example at 37° C. Reduction of solvent in the droplet can also be achieved by addition of another solvent to the oil phase with higher water solubility (e.g., hexane, ethanol, methanol).

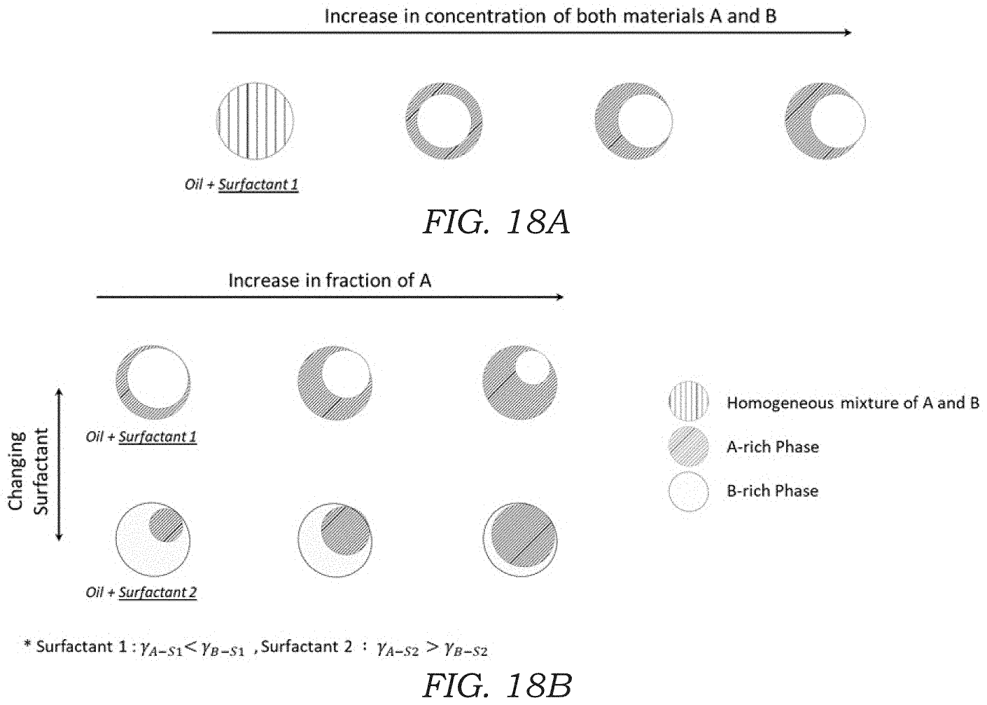

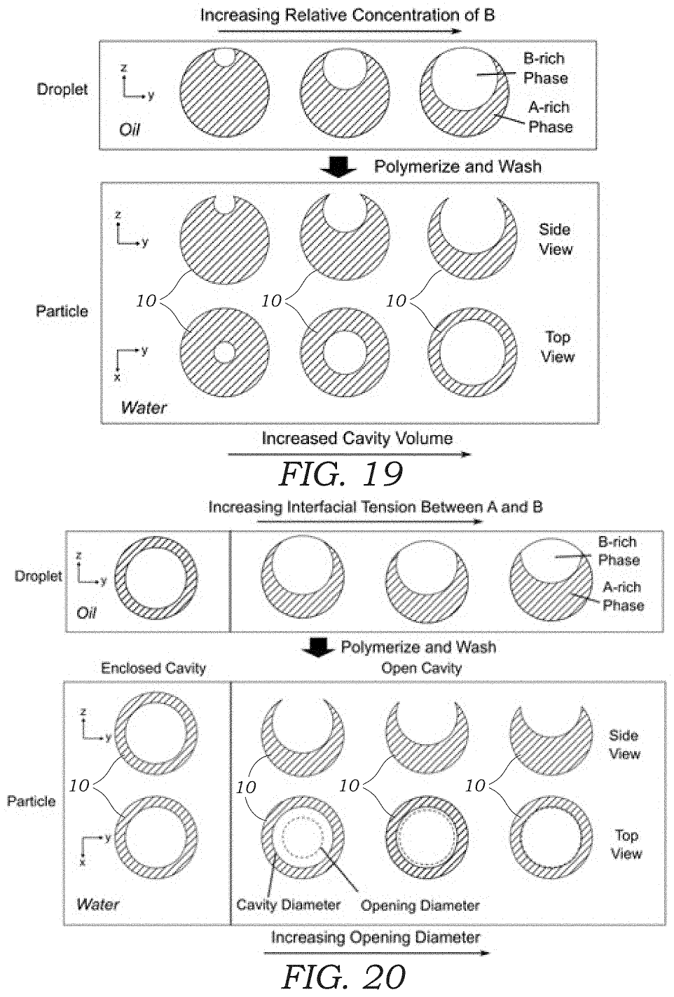

The change in one or more of these conditions also regulates the internal shape of the separated phases by shifting the balance among miscibility and interfacial tensions between the two separated phases and the interfacial tensions between the separated phases and the surrounding dispersion media. It is beneficial to be able to induce different shapes of droplets since it potentially extends the applications where the phase-separated droplets can be used. In some embodiments, the final configuration of the different phases can be changed by adjusting the interfacial tensions and by adjusting the relative concentration of each polymer (FIG. 19). Interfacial tensions can be adjusted via a number of methods. For example, changing the concentration or types of surfactants used, or adjusting the total concentration of precursor materials. Examples of this are depicted in FIGS. 18A-18B. FIG. 18A depicts the effects of precursor concentration on the multi-phase droplet morphology. In this example, the two polymer phases undergo phase separation above a critical concentration. Here the interfacial tension between the oil phase and the A-rich phase is lower than the interfacial tension between the B-rich phase and the oil phase. At some concentrations the A-rich phase may completely surround the B-rich phase to minimize the system energy. As concentration is increased the interfacial tension between A and B is increased while the ratio of interfacial tension between A-oil and B-oil does not change. In this situation the inner phase may wet the oil phase to minimize energy. A continued increase in the total polymer concentration can slightly deform the spherical shape of the droplets. In another example, the volume of each phase can change depending on the ratio of the precursor materials (FIG. 18B). For example, to achieve a greater volume of the A-rich phase, we can increase the concentration of the A precursor material in the precursor solution. In another example, the droplet morphology can be adjusted by changing the concentration of surfactant or type of surfactants. Consider surfactant 1, which has a lower interfacial tension of the oil with polymer A then with polymer B. For this system the A-rich phase will preferably occupy the oil interface. Similarly, surfactant 1, which has lower interfacial tension of oil with polymer B would result in the opposite configuration where the B-rich phase preferably occupies the oil interface and the A-rich phase forms a cavity or enclosed region.

How a certain surfactant behaves can depend on a number of properties. Significantly, ionic and non-ionic surfactants can have very different interfacial tensions with different types of polymers. FIGS. 19 and 20 describe the morphologies following induced phase separation depending on concentration of the precursor materials in the A-rich and B-rich phases (FIG. 19) and interfacial tension between the A-rich and B-rich phases (FIG. 20) and also indicate the final polymerized shaped particles resulting from these phase-separated morphologies. The shaped particles 10 span a family of shapes in which a spherical void or cavity 12 is subtracted from an outer spherical envelope. In some embodiments the cavity opening diameter is smaller than the void or cavity 12 diameter, while in other embodiments the cavity opening diameter is of substantially equal size to the void or cavity 12 diameter as shown in FIG. 20. A cavity opening diameter that is smaller than the void or cavity 12 diameter is preferred in some embodiments described herein to reduce the diffusive and convective loss of biomolecules released from cells 50 or to reduce the fluid shear stress on cells 50 adhered within the cavity.

Polymerization to Form Shaped Particles

In some embodiments, following the induced phase separation of droplets containing precursor materials using approaches described herein, the phase separated droplets are exposed to a stimulus to crosslink a phase separated precursor material to form a shaped particle 10. Depending on the precursor material chemistry a variety of methods can be used to initiate crosslinking following induced phase separation. Crosslinking includes covalent bonding, ionic bonding, molecular entanglement, hydrogen bonding, hydrophobic interaction and crystallite formation.

In one embodiment, droplets including one or more photopolymerizable materials are crosslinked to form shaped particles 10 by exposure to UV or visible light. Photopolymerization generally uses a photoinitiator that has high absorption at a specific wavelength of light to produce radical initiating species. Exemplary photoinitiators are described in the Precursor Materials section herein. Photopolymerizable materials include PEG acrylate derivatives, PEG methacrylate derivatives, polyvinyl alcohol (PVA) derivatives, and modified polysaccharides such as hyaluronic acid derivatives and dextran methacrylate. Also, multifunctional thiols and alkenes, such as DTT and PEG-norbornene, that are photo-crosslinkable through a thiol-ene reaction are considered as photopolymerizable materials. The radiation source includes a mercury vapor arc (220-320 nm), a tungsten filament (300-2500 nm), a deuterium arc lamp (190-400 nm), xenon arc lamp (160-2,000 nm) and light emitting diodes (LED) for the visible wavelengths (360-900 nm). In one embodiment, the visible light has a range of about 380-650 nm. In one embodiment, UV light has a range of about 300-400 nm. In one embodiment, the precursor solution is irradiated with light for between about 1 seconds and 30 minutes to photocrosslink the combined solution. In one embodiment, the power of the light source has a range of about 1-300 mW/cm². The total energy delivered for polymerization may range between ˜10 mJ/cm² to 10,000 mJ/cm². In one exemplary embodiment, phase-separated droplets, containing aqueous solution of 7.5% w/w 2-arm PEG-acrylate 1500 Da, 15% w/w fish gelatin and 0.5% w/w LAP, are crosslinked in a 4° C. refrigerator with a 20 W LED lamp (395-400 nm) for 10 min. The distance from lamp to the droplet solution is about 5 cm.

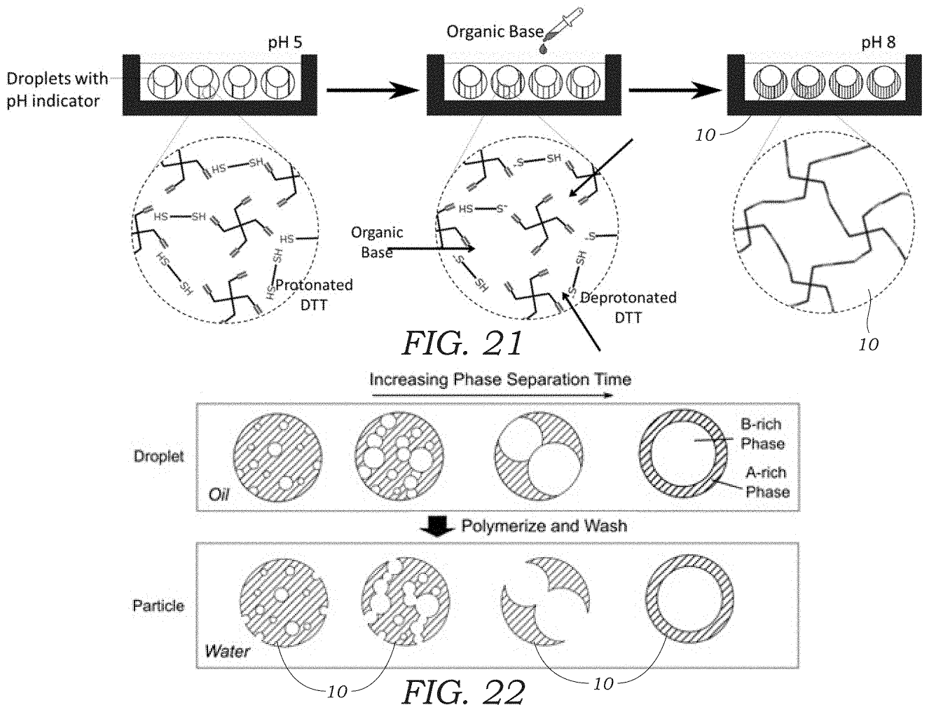

In addition to polymerization by exposure to light, other stimuli can be introduced to convert droplets into shaped particles 10. In one embodiment, when the crosslinking kinetics of precursor materials is highly dependent on pH, polymerization of droplets is initiated by a change in pH. For example, an increase in pH deprotonates a thiol and it acts as a nucleophile and donates an electron pair to form a covalent bond with different functional groups including 4-fluorophenyl group, vinyl sulfone group, maleimide group and pyridyl disulfide group. In one exemplary embodiment, as described in FIG. 21, droplets with aqueous solution of 7.5% w/w 4-arm PEG-VS 1500 Da, 15% w/w fish gelatin and 0.005% w/w DTT are generated at room temperature and dispersed in Novec™™ 7500 fluorinated oil with 0.5% Pico-Surf™ surfactant. To characterize the pH of the droplets, a pH indicator can be added to the precursor solution. The droplets are initially at pH 5 such that the thiol groups on the crosslinker molecules remain predominantly protonated, inhibiting the Michael addition reaction. After the droplets are kept in a 4° C. refrigerator for 2 hour to induce phase separation, various amount of Novec™ 7500 oil with 3% v/v triethylamine (Sigma) is introduced into the droplet suspensions to increase the pH of the aqueous phase to around pH 8.1, deprotonating the thiol groups and initiating the crosslinking of droplets.

Preferably, to make shaped particles 10, the phase-separated droplets include at least one of crosslinkable phase that is rich in crosslinkable precursor materials and one or more non-crosslinkable phases where the concentration of crosslinkable materials does not cause polymerization. The polymerization stimuli for droplets should not enable the crosslinking of the non-crosslinkable phase so that precursor materials in the non-crosslinkable phase do not crosslink or have a polymerization process that is kinetically unfavorable compared to the polymerization of materials in the crosslinkable phase. In some embodiments, after the polymerization step, precursor materials from the non-crosslinkable phase are incorporated in the crosslinked polymer network at the interface between the crosslinkable phase and the non-crosslinkable phase. The incorporation of materials from a non-crosslinkable phase can be achieved due to physical entanglement or chemical reactivity with materials in the crosslinkable phase. This process leads to the interface surface having a different chemical functionality than other surfaces of the crosslinked shaped particle 10. Having a surface of the polymerized shaped particle 10 with chemical groups different from the main body of the particle can add functionality to the particle 10, such as a localized cell adhesive region 14 and/or localized affinity capture agent 16. For example, when shaped particles 10 are made from aqueous droplets composed of 4-arm PEG-acrylate-rich phase and gelatin-rich phase, the gelatin molecules at the interface are embedded in the PEG hydrogel after crosslinking (FIGS. 3A-3D), which can promote cell adhesion in the following steps through interaction between cells 50 and cell adhesive moieties in gelatin. Selective cell attachment to the localized cell adhesive region 14 or layer is useful for applications such as single cell 50 secretion screening where a cell 50 should adhere to the inner surface but not to the outer surface of a crescent shaped particle 10 (FIG. 1A, FIG. 7B). Alternatively, to incorporate more gelatin, some or all the gelatin components can be modified with reactive groups that can crosslink with PEG during the droplet polymerization step. The concentration of modified gelatin should be low enough to prevent crosslinking between gelatin molecules in the non-crosslinkable phase while gelatin can crosslink to the PEG which is abundant at the interface. The modified gelatin includes but is not limited to gelatin methacryloyl (GelMA), allyl modified gelatin (e.g., pentenoyl gelatin), alkyne functionalized gelatin and thiol-modified gelatin.

In addition to previously discussed crosslinking approaches, temperature may be used to polymerize one or more phases following induced phase separation. For example, agarose or gelatin can be physically crosslinked by lowering temperature. This approach can be particularly useful for applications where it is desired to dissolve the particle at a later point by adjusting temperature.

In some embodiments, polymerization is initiated prior to completion of the induced phase separation process. In this case, smaller domains of the separated phases have formed randomly throughout the droplets but they have not coalesced to an equilibrium state. Polymerization of a precursor material in this state can generate porous polymer shaped particles 10 (FIG. 22). The time between triggering of induced phase separation and polymerization can be controlled to control the relative size of the pores within the polymerized shaped particles 10 (FIG. 22). Preferably, in this embodiment the induced phase separation is triggered to have a slow time constant for phase separation to enable capturing of the intermediate state of phase separation during polymerization. For example, using a smaller temperature change from the initial mixed condition, or conditions resulting in a state closer to the boundary of the binodal curve (FIG. 5).

Use of Shaped Particles

Polymerized shaped particles 10 manufactured as described herein can be used for a number of applications disclosed herein. In addition, shaped particles 10 manufactured with related techniques can be applied to the inventive uses and applications disclosed herein. Thus, shaped particles 10 manufactured or formed by other methods can be used in the inventive methods of use described herein.

Shaped particles for culture and analysis of adherent cells. In one embodiment, hydrogel-based shaped particles 10 containing voids or cavities 12 open to an external fluid are used to adhere cells 50, such as stem cells, for culture in large batches. Hydrogel-based shaped particles 10 with a crescent-shaped cross-section are functionalized during manufacture with cell adhesive moieties (e.g., arginine-glycine-aspartate, RGD peptides, poly-l-lysine, gelatin or a fragment thereof, collagen or a fragment thereof, or other adhesive moieties known in the art). In a preferred embodiment, RGD peptide (Ac-RGDSPGERCG-NH2) [SEQ ID NO: 1] or another integrin binding peptide or derivative peptide can be incorporated into the precursor solution and covalently crosslinked into the polymer backbone through reaction with a cysteine group present within the peptide. Cells 50 can be seeded onto shaped particles 10 settled on the bottom of a well plate or other vessel or mixed with shaped particles 10 in solution to adhere predominantly in the voids or cavities 12 of the shaped particles 10. Once adhered, cells 50 can spread and proliferate on the shaped particles 10. Shaped particles 10 can be used to grow adherent cells 50 in large stirred tank bioreactors. The void or cavity structure 12 can protect adhered cells 50 from fluid shear stress in the reactor. For example, the void or cavity 12 can reduce fluid shear stress by greater than an order of magnitude compared to the outside of the shaped particle 10 on adhered cells 50. This can allow for improved cell function in stirred tank bioreactors. In addition, the void or cavity 12 of the shaped particle 10 can reduce fluid shear stress during processing of adherent cells 50 during pipetting, or other fluid transfer steps associated with high flow/shear stress. In addition, the void or cavity 12 of the shaped particle 10 can reduce fluid shear stress during processing of cells 50 attached to shaped particles 10 in a flow cytometer or fluorescence activated cell sorter (FIGS. 7E, 11). Cells 50 attached to shaped particles 10 can be passaged by mixing with additional empty shaped particles and allowing the plurality of shaped particles to settle in a vessel to form a settled aggregate. Adherent cells 50 can then grow and migrate to adjacent shaped particles 10 during this settled period and transfer to a larger surface area to expand the culture. Preferably, the vessel is coated to be non-adherent to cells. The batch of shaped particles 10 can be stirred again in a bulk bioreactor. Cells 50 seeded onto shaped particles 10 can be used for additional applications as discussed herein.

Capture of biomolecules released by cells/cell secretion assays. In one embodiment, shaped particles 10 containing voids or cavities 12 open to an external fluid are used to adhere cells 50 and capture biomolecules released from the cells 50. Illustrative examples of such cell secretion assays are seen in, for example, FIGS. 9A-9H, 25A-25G, FIGS. 26A-26D, FIGS. 27A-27C. Shaped particles 10 with a crescent-shaped cross-section are functionalized during manufacture with one or more affinity capture agents 16. These may include, for example, cell adhesive moieties (e.g., arginine-glycine-aspartate, RGD peptides, gelatin or a fragment thereof, collagen or a fragment thereof, poly-l-lysine, or other adhesive moieties known in the art) and an affinity capture moiety (e.g., biotin, antibody, antibody fragment, aptamer, minibody, nucleic acid, oligonucleotide, mRNA capture probe). In a preferred embodiment, RGD peptide ([SEQ ID NO: 1]) or another integrin binding peptide or derivative peptide can be incorporated into the precursor solution and is covalently crosslinked into the polymer backbone through reaction with a cysteine group present within the peptide. In a preferred embodiment 5 mM of the RGD peptide is incorporated into the precursor solution. In another preferred embodiment, shaped particles are biotinylated through the incorporation of biotin PEG-thiol within the precursor solution composed of PEG and gelatin in the microfluidic droplet generation device 20. In another preferred embodiment, shaped particles 10 are biotinylated through the incorporation of gelatin-functionalized with biotin within the precursor solution composed of PEG and gelatin in the microfluidic droplet generation device 20. Biotinylation of shaped particles 10 enables conjugation of many common reagents, such as antibodies, proteins, nucleic acids, cells, nanoparticles, and primers, and allows for their use in a wide range of assays including ELISA, PCR, and flow cytometry.

In one embodiment the cell concentration is tuned with respect to the shaped particle 10 concentration such that a majority of cell-occupied shaped particles 10 contain a single cell 50, i.e., following a Poisson distribution. In a related embodiment, the size of the void or cavity 12 within the shaped particles 10 are dimensioned to hold only a single cell (e.g., void or cavity diameters of 10-30 micrometers depending on the cell size). After incubating cells 50 with shaped particles 10 to allow adhesion, often 1-2 hours, a biomolecule capture moiety is added to bind to the shaped particle surface, e.g., through biotin-streptavidin non-covalent linkages. In other embodiments a biomolecule capture moiety may be added to the shaped particles 10 prior to cell 50 seeding. In some embodiments, cells 50 attached to shaped particles 10 are then incubated to allow secretion and capture of secretions on the shaped particles 10 using the one or more affinity capture agents 16. Reduction in convective and diffusive loss of secretions can be achieved by incubating shaped particles 10 with cells 50 in a solution which has a low permeability to the secretions, such as Ficoll, dextran, alginate, agarose, gelatin or the like. After incubation, cells 50 are then washed and shaped particles 10 are stained with a secondary labeling agent that binds to the secreted product and contains a fluorescent dye, magnetic particle, oligonucleotide barcode, metal isotope barcode, or a combination of labels listed herein.

In related embodiments, cells 50 attached to shaped particles 10 are lysed to release biomolecules which are then captured by the affinity capture agent 16 on the shaped particle 10. For example, the cells 50 may be lysed to release mRNA that is captured locally on the shaped particle 10. The local capture of released biomolecules, like mRNA, is facilitated by the enclosed void or cavity 12 of the shaped particle 10 which reduces diffusion out of the smaller shaped particle opening and limits convection of fluid and mass transport out of the void or cavity 12. Lysis can be achieved for example, using a detergent or surfactant (e.g., non-ionic detergents such as Triton X-100, Tween 20, etc. or ionic detergents such as sodium dodecyl sulfate, Sarkosyl, etc.), a temperature increase, or a combination of the above. Detergent or surfactant solution can be introduced to shaped particles 10 settled on the bottom of a well plate using gentle pipetting to avoid convective loss of biomolecules from cells 50. Further reduction in convective and diffusive loss can be achieved by seeding shaped particles 10 with cells 50 in a higher viscosity solution, such as Ficoll, dextran, alginate, agarose, or the like, prior to addition of the surfactant or detergent solution. Following lysis and capture on the hydrogel material of biomolecules from the lysed cell, the remaining lysis solution can be washed away and additional reactions or analyses can be performed. For example, mRNA captured by poly-T capture oligonucleotides on the surface of the shaped particle 10 can be reverse transcribed using reverse transcriptase enzyme and the cDNA can be encoded with a barcoded nucleic acid sequence incorporated into the capture oligonucleotides. In some embodiments, each shaped particle 10 can contain a unique barcoded oligonucleotide sequence associated with the poly-T capture sequence. These unique sequences can be obtained using split and pool stepwise oligonucleotide synthesis approaches known in the art. This particle-specific oligonucleotide barcoding enables the mRNA released from the individual cells 50 captured in each shaped particle 10 to be separately identifiable in a downstream pooled cDNA sequencing process.

Shaped particles 10 can be dimensioned for use with flow cytometers or fluorescence activated cell sorters (FACS). Preferably, shaped particle diameter is <100 micrometers, and more preferably <70 or <40 micrometers. In some cases, when large particle cell analyzers or microscopy are used for analysis, the shaped particle diameter is preferably <200 micrometers or <150 micrometers. Shaped particles 10 with or without attached cells 50 may be analyzed and sorted using commercial flow cytometer instruments including On-Chip Sort, Biosorter, BD FACSAria Sony SH800. The shaped particles 10 can act as a cell-carrier to allow adherent cells 50 to be analyzed in flow cytometers and FACS instruments without the need to bring the adherent cells 50 into suspension which can be damaging to the cytoskeletal structure of these cells 50 and their underlying function. Cells 50 adhered within the protected void or cavity 12 of these shaped particles 10 can also experience reduced shear stress from the flow around the shaped particle 10 and have improved viability after sorting (FIGS. 7E, 11). Shaped particles 10 with asymmetric shapes can exhibit preferred orientations in flow which can improve the repeatability of measurements and further reduce shear stress. High fluid shear stress can damage sensitive cells 50 in FACS sorting and ultimately reduce cell viability or alter function. When performing flow cytometric analysis and sorting of crescent-shaped particles 10 the following protocol can be used. A suspension of shaped particles 10 is introduced into the input well or vial for the flow cytometer. The flow cytometer is started to apply pressure/drive flow of the shaped particle 10 containing solution and analyze shaped particles 10 one-by-one in the optical analysis region of the flow cytometer. Optionally, shaped particles 10 with a desired signal (forward scatter, side scatter, or fluorescence) can be selected and sorted. The timing delay for the flow sorter may be adjusted to tune it for the larger size of the shaped particle 10 compared to the cells 50 that are normally analyzed in these systems. Preferably, the flow cytometer or FACS instrument is operated at rates of >100 shaped particle events per second, more preferably >500 shaped particle events per second. For flow cytometry or FACS-based analysis, preferably >10,000 shaped particles 10 containing cells 50 are analyzed or more preferably >20,000, >50,000, or >100,000 shaped particles 10 containing cells 50, to analyze large populations to identify rare cells 50. For analysis with single-cell sequencing instruments, such as 10× Chromium, BD Rhapsody, Mission Bio Tapestri, preferably >1,000 shaped particles 10 containing cells 50 are analyzed, or more preferably >5,000 or >20,000 shaped particles 10 containing cells 50 are analyzed.

Exemplary Workflow for Using Shaped particles to Capture Biomolecules Released from Cells

(1) Loading Cells on the Shaped Particles in an Aqueous Solution.

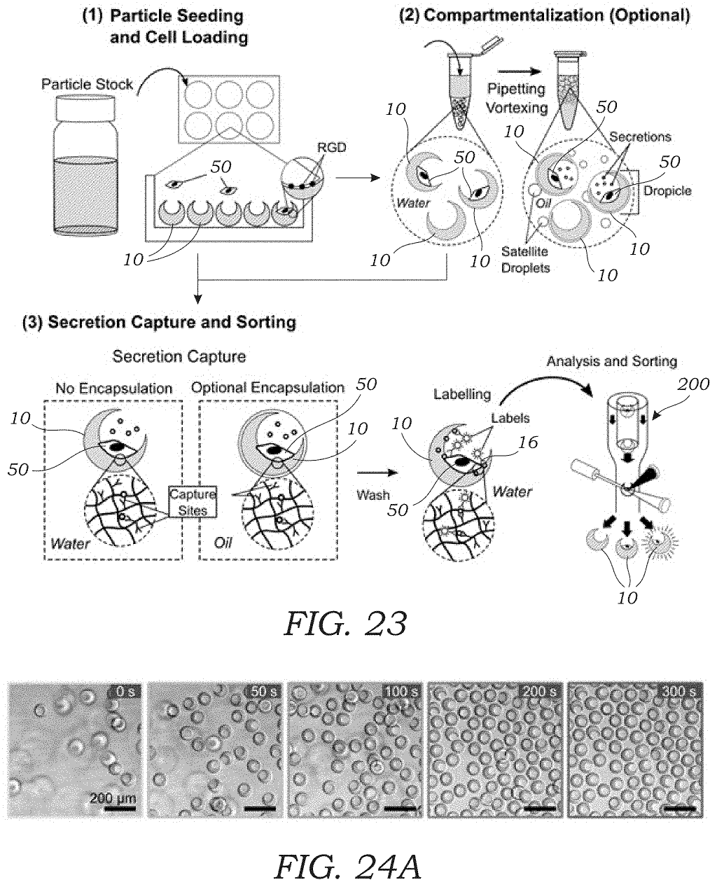

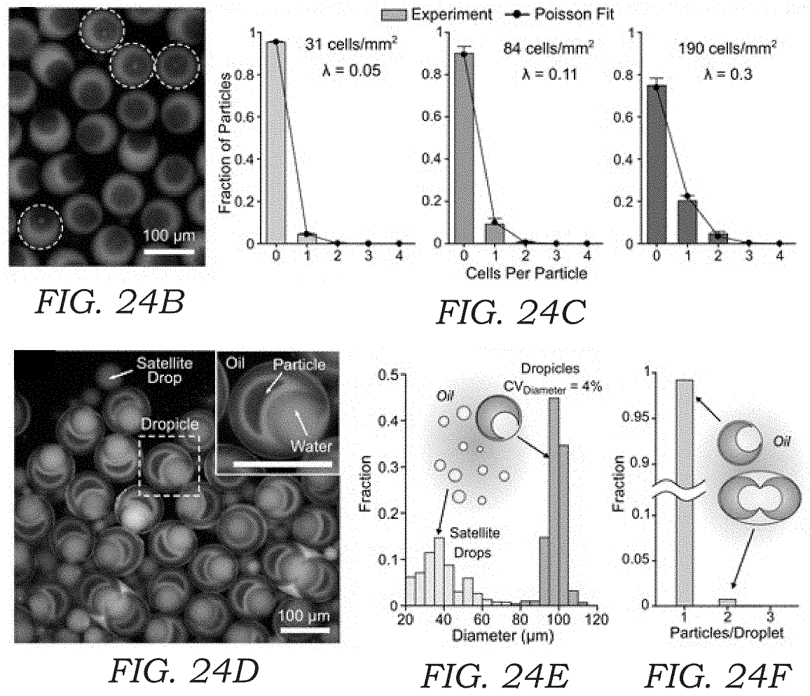

In one example embodiment, shaped particles 10 including a void or cavity 12 open to the surrounding environment, and having cell-adhesive moieties, and further including affinity capture agent(s) 16, are first loaded, e.g., by pipetting, into a well plate, well, flask, or other vessel with a flat bottom surface (See FIG. 23—Particle Seeding and Cell Loading). Due to the asymmetry of the shaped particle 10 shape in some embodiments, shaped particles 10 settle with their respective voids or cavities 12 located in an upright orientation (e.g., the void or cavity 12 opens to the surface opposite the direction of acceleration due to gravity) (FIG. 24A). This is advantageous in that the open void or cavity 12 can then be seeded with cells 50 (FIG. 24B). The number of shaped particles 10 to seed can be approximated for a given particle diameter and well surface area by assuming closed packing of spheres. For example, for a particle diameter of 85 microns and a twelve well plate (surface area 2 cm² per well) it was found that microliters of concentrated particles 10 covered a large fraction of the bottom of the well surface.

After shaped particles 10 settle (typically 5-10 mins), cells 50 can then be carefully seeded into the wells (e.g., using a pipette) and allowed to settle, with a fraction of the cells 50 settling in the voids or cavities 12 of the shaped particles 10 (FIG. 23). The fraction of cells 50 that fall into the voids or cavities 12 vs external to the voids or cavities 12 can be increased by increasing the ratio of the void or cavity opening width to the shaped particle diameter. In general, the fraction of shaped particles 10 containing a given number of cells 50 (i.e., 0, 1, 2, 3, 4, 5, 6, 7, 8, 9, 10, or more cells) can be calculated from Poisson statistics, and can be controlled by adjusting the cell density during seeding stages. In one example experiment, 15 microliters of concentrated shaped particles 10 (85-micron outer diameter, 50-micron inner diameter) were seeded in each well of a twenty-four well plate. Various amounts of cells 50 were added to different wells and allowed to settle into the shaped particles 10. Cells 50 and shaped particles 10 were imaged to determine the number of cells per particle. Addition of 10,000 cells 50 per well resulted in Poisson loading of approximately 0.09 cells per particle. More specifically, 93.1% of shaped particles 10 were empty, 6.65% contained one (1) cell, and 0.25% contained two (2) or more cells 50. Of shaped particles 10 with cells 50, 96% contained only one cell 50. Addition of 30,000 cells per well resulted in Poisson loading of approximately 0.2 cells per particle. More specifically, 82.2% of shaped particles 10 were empty, 15.9% contained one (1) cell, and 1.9% contained two (2) or more cells 50. Of particles with cells 50, 89% contained only one cell 50. Addition of 100,000 cells per well resulted in Poisson loading of approximately 0.8 cells 50 per shaped particle 10. More specifically, 42.6% of shaped particles 10 were empty, 32.6% contained one (1) cell 50, and 24.8% contained two (2) or more cells 50. Of shaped particles 10 with cells 50, 56.8% contained only one (1) cell 50.

Different cell seeding amounts are ideal for different applications. Generally, higher cell seeding densities result in shaped particles 10 having more than one cell 50 per particle. See e.g., FIG. 24C. For embodiments in which capturing no more than one cell per particle is critical for assay validity, lower seeding densities are ideal (e.g., 10,000 cells per twenty-four well plate well (surface area of 2 cm²)). Alternatively, the size of the void or cavity 12 in the shaped particles 10 can be tuned to enrich for single cell 50 loading surpassing Poisson statistics (FIG. 7C, FIGS. 10A-10C). In contrast, for embodiments in which multiple cells per particle are desirable, such as for evaluation of cell-cell interactions a higher seeding density is preferred (e.g., 100,000 cells per twenty-four well plate well (surface area of 2 cm²) in which 15 μl of concentrated shaped particles 10 in a twenty-four well plate were seeded in each well). In other embodiments, where loss of cells 50 is detrimental and should be limited, multiple layers of particles 10 can be arrayed such that cells 50 that do not settle into the voids or cavities 12 of the first layer of particles 10 can settle into voids or cavities 12 in second or subsequent layers of particles 10.

A second method of associating cells 50 with shaped particles 10 leverages the difference in shear forces experienced by cells 50 bound within sheltered shaped particle voids or cavities 12 versus those bound on outer surfaces of shaped particles 10. In brief, a concentrated suspension of cells 50 with affinity to the surface of the shaped particle 10 (e.g., through adhesive ligands) can be mixed into a concentrated suspension of shaped particles 10 and agitated vigorously. Cells 50 will distribute throughout the suspension and rapidly adhere to the surfaces of the shaped particles 50. Those that bind to the exterior of the shaped particle 50 will be sheared off rapidly and return back into suspension, whereas cells 50 which become entrapped within the void or cavity 12 are more sheltered from much of the external fluid shearing force, leaving them adhered to the surface of the pocket formed by the void or cavity 12 of the shaped particle 10. Once particle suspensions have been sufficiently agitated, they can be filtered as described below. This offers a method to rapidly enrich the fraction of cells 50 found within the shaped particle void or cavity 12. This method is facilitated by a localized cell adhesive region 14 leading to stronger binding of cells within the void or cavity 12 compared to outer regions of the shaped particle 10.

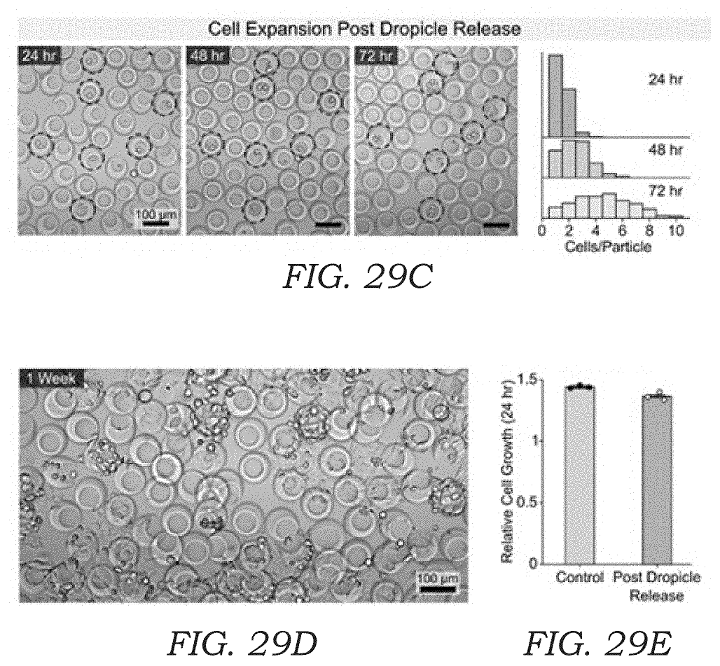

Modification of the surfaces of shaped particles 10 enables adhesion and subsequent culture of seeded cells 50 within shaped particle voids or cavities 12. For example, commonly used integrin binding peptides, such as RGD, incorporated into the surface of the shaped particle 10 enables adhesion of cells 50 for example, based on the presence of integrins, maintaining the attachment of cells 50 to the shaped particles 10 even in the presence of vigorous mechanical agitation from pipetting, centrifugation, and flow sorting procedures. In a preferred embodiment, RGD is added at a concentration of at least 4 mg/mL in the precursor materials solution containing PEG norbornene during shaped particle manufacture. In this approach, radicals generated from photoinitiators in the PEG phase induce covalent bonding between free thiols on peptide cysteine groups and unbound norbornenes on the polymer backbone of the shaped particle precursor. CHO cells 50 seeded on such RGD-modified shaped particles 10 remained associated and spread on the surface of the shaped particle 10 for several days (FIGS. 29C, 29D).

Cell lines which are typically non-adherent can also be associated with surfaces of the shaped particle 10. In one embodiment, biotin-streptavidin interactions are used to link cells 50 to shaped particles 10. More specifically, biotinylated shaped particles 10 are pre-modified with streptavidin and target cells 50 are pre-modified with biotin (e.g., biotin NHS), biotinylated lipids/cholesterols or biotinylated antibodies generating affinity between shaped particles 10 and cell populations or subsets of cell populations. In one preferred workflow, primary T-cells 50 can be bound to biotinylated shaped particles 10 by first pre-modifying biotinylated shaped particles 10 with ten (10) μg/mL of streptavidin in PBS. Concurrently T-cells 50 are modified by mixing 10 μg/mL of biotin-anti-CD3 and/or anti-CD45 antibody to fewer than 10 million cells, and incubated at 37° C. for 20 minutes. Both shaped particles 10 and cells 50 are washed several times with PBS to ensure removal of unreacted groups unbound materials. Shaped particles 10 are then spun down for 5 minutes at 2000 G to form a tight pellet to which a concentrated anti-CD3 and/or anti-CD45 modified cell suspension is added. The cell and suspension of shaped particles 10 is then continuously agitated by manually pipetting for at least 2 minutes. The sample is then filtered using a cell strainer, as described below, to collect only shaped particles and any cells 50 that were bound to their surface. An alternative embodiment using biotinylated lipids or NHS-biotin proceeds in much the same way, with the important caveat that any cell 50 can be modified using this approach, regardless of surface protein composition. Here, cells 50 are incubated at 37° C. with 10-100 μg/mL of biotinylated lipids or NHS-biotin to bind to cell-surface proteins for a total period of 60-90 minutes before washing and attaching to shaped particles 10 by pipetting. Alternatively, capture antibodies such as anti-CD3 and/or anti-CD45 are conjugated directly or linked through biotin/streptavidin binding to the shaped particles 10, followed by loading T-cells into the shaped particles 10.

(2) Washing Away Unbound Cells and/or Background Secretions and Adding an Affinity Capture Agent to Bind to the Shaped Particles that Captures a Specific Biomolecule of Interest Released from Cells.

In certain applications, cells 50 that remain unassociated with shaped particles 10 are undesirable and may even become a source of noise. In order to reduce background, shaped particles 10 can be washed prior to incubation, eliminating unbound cells 50 from the solution before assays are conducted. In one approach, the suspension of shaped particles 10, shaped particles 10 with attached cells 50, and unassociated cells 50 are added to a cell strainer with a mesh size larger than the cell diameter but smaller than the shaped particle diameter. This allows shaped particles 10 with attached cells 50 to be retained by the mesh, while unassociated cells 50 pass through. While the shaped particles 10 are retained, they can be continuously washed by sequential additions of buffer, eliminating any cells 50 not tightly adhered to the surface of the shaped particles 10. Shaped particles 10 and their associated cells 50 are subsequently isolated through simple inversion of the cell strainer, addition of buffer from the underside of the mesh, and collection of the resulting solution containing buffer and eluted shaped particles 10 with attached cells 50. One preferred cell strainer for this application is the Fisherbrand 40 μm sterile cell strainer from Fisher Scientific.

In applications with rare cells 50, it may be desired to recover any cells 50 not associated with the shaped particle voids or cavities 12. In this case, the above strategy can be used, with cells 50 not associated with shaped particles 10 collected for later seeding into a new sample of shaped particles 10.

In some applications it is desired to capture biomolecules released from cells 50, such as cell secretions or released nucleic acids, onto shaped particles 10. In such embodiments it is beneficial to modify the surface of shaped particles 10 with an affinity capture agent 16 such as an antibody or fragment thereof or immunoglobulin binding proteins to act as a molecular capture site for secretions (FIG. 9A and FIG. 23—Secretion Capture and Sorting). In exemplary embodiments in which the rate of secretion is slow and the binding of cells 50 to shaped particles 10 is rapid, as in biotin-streptavidin reactions or antibody/antigen interactions, shaped particles 10 can be pre-modified with molecular capture sites internal or on the surface or within the cell adhesive region 14 of the shaped particle 10 without contaminating signals stemming from the associated cells 50. Also, for applications when biomolecules are not released from cells 50 until a stimulus (e.g., cell lysis reagents are added), pre-modification of capture sites is preferred. In other embodiments, where secretion rates are rapid and cells 50 may take several hours to strongly adhere to the surfaces of shaped particles 10, as in the case where adherent cells 50 (e.g., CHO cells) are adhered to an RGD binding peptide, cells 50 are first allowed to adhere to surfaces of the shaped particles 10, preferably for between 2-12 hours. Once cells 50 have attached to the shaped particles 10, samples can be washed via centrifugation and solution exchange to remove background secretions and unbound cells 50. Shaped particles 10 can then be modified with affinity capture agents 16 such as biotinylated protein A or biotinylated antibodies that bind to secretions/proteins of interest to form molecular capture sites attached to the shaped particles 10.

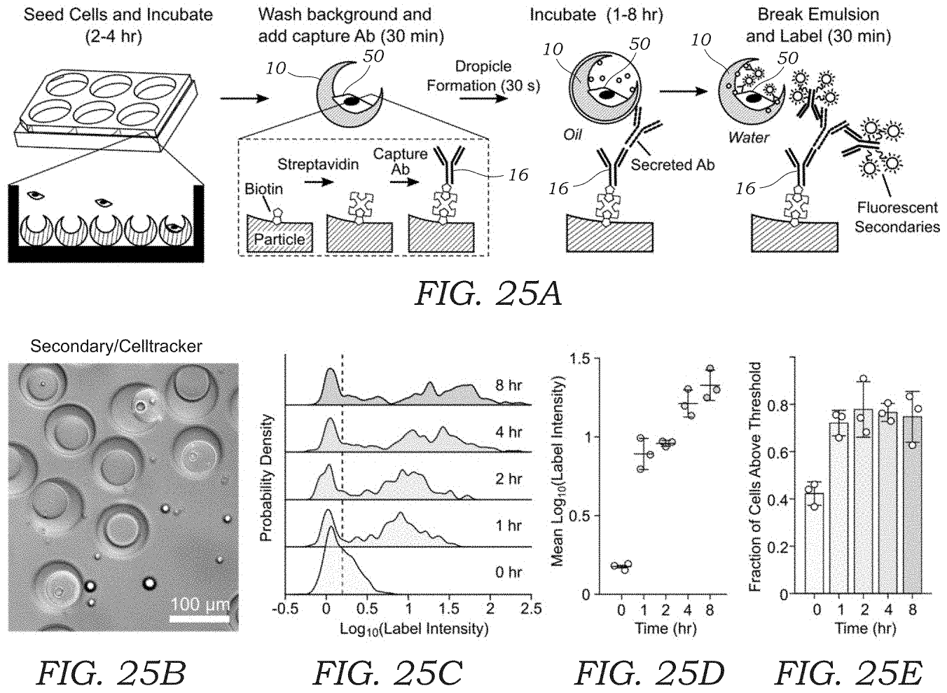

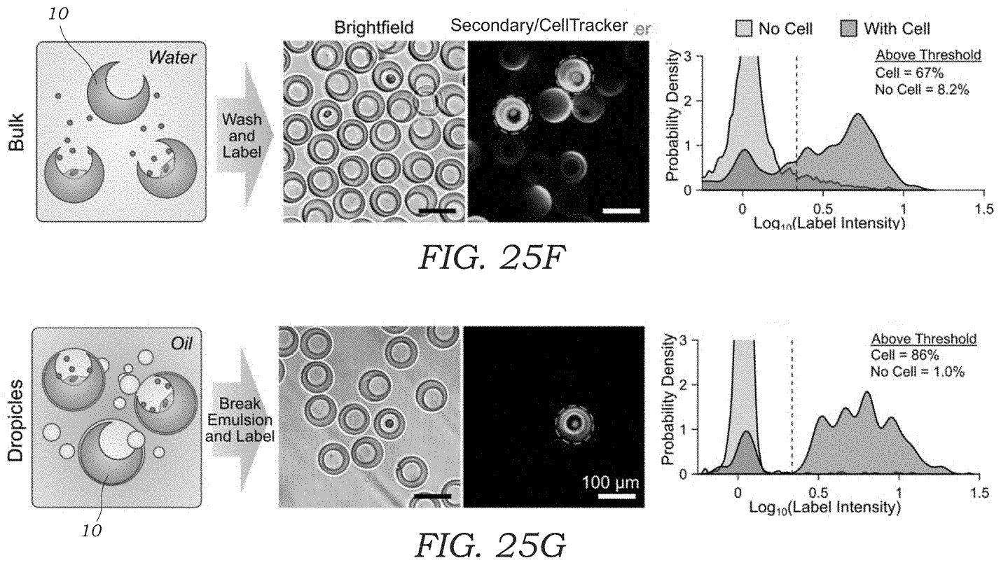

A detailed protocol is disclosed herein. In one preferred embodiment, shaped particles 10 are functionalized with both RGD peptides and biotin groups by incorporating both 4 mg/mL RGD and 0.5-5 mg/mL biotin-PEG-thiol in the precursor material solution. Cells 50 are seeded on these peptide and biotin modified shaped particles 10. After attachment, each shaped particle sample (30 μL of shaped particles in a twelve well plate) are treated with 0.02 mg/mL streptavidin in PBS, which binds to biotin groups on surfaces of the shaped particle 10, incubated for 10 minutes, and washed several times with PBS with 0.5% bovine serum albumin (BSA). Next, each concentrated particle sample is modified with 10 μL of a 0.5 mg/mL stock of biotinylated-protein A and incubated for ten minutes, as described above. Alternatively, biotinylated anti-IgG or other antibodies can be incubated and bound (FIGS. 25A-25G and 26A-26D, 27A, 27B). After appropriate modification, shaped particles 10 can be directly applied for secretion measurement applications.

(3) Incubating the Shaped Particles for a Time Period to Accumulate Released Biomolecules, Such as Secretions, that Bind with the Affinity Capture Agent.

Analytes, such as secretions or biomolecules released from single cells 50 within shaped particles 10 are generally retained within and bind to the surface of the shaped particle 10 during incubation (FIG. 25F), although a small amount of analytes can be released through the shaped particle 10 opening to the surrounding solution. Potential cross-talk to neighboring shaped particles 10 can be avoided by seeding particles 10 at a lower density, such that the distance between neighboring particles 10 is on average greater than five (5) shaped particle diameters, or even greater than ten (10) shaped particle diameters. Depending on expected secretion levels, incubation times can be adjusted depending on cell type and secretion targets of interest. For example, if low levels of secretions are expected longer incubation times can be used (>12 hours); in the case of high secretion rates shorter incubation times can be used (˜2-3 hours, or even 30 minutes). Crosstalk can also be reduced by adding components to the incubation solution to increase viscosity and thereby decrease diffusive transport and convective transport away from the shaped particle 10. Example components include, but are not limited to: Ficoll, dextran, alginate, agarose. Concentrations of the viscosity increasing components are preferably ˜2-20% w/w in aqueous solution. As explained herein, crosstalk can also be reduced by using shaped particles 10 with affinity capture agents 16 localized within a void or cavity 12 (FIGS. 9D-9H, FIGS. 12A-12B).

Depending on the expected secretion levels, the number of secretion binding sites and or spatial location of binding sites can be adjusted. In one example, available binding sites can be increased by fabricating shaped particles 10 with a matrix porosity with pore sizes that allow secretions to freely diffuse through the gel matrix. In this embodiment, the full 3D geometry of the shaped particle 10 can be used to capture secreted molecules, increasing the total number of binding sites, which is beneficial for high secretion levels as binding sites are not easily saturated enabling better dynamic range of detection. In some cases, e.g., low secretion levels, it is advantageous to spatially localize the binding sites in order to create a more concentrated signal. For example, by using solid shaped particles 10, or particles 10 with porosity such that secretions cannot freely diffuse through the matrix of the shaped particle 10, accessible binding sites are localized to the surface of the shaped particles 10. In further embodiments, binding sites can be localized to the surface of the inner void or cavity 12 to further localize the secretion capture and resulting signal (FIG. 9E). In the detailed example description of CHO cells 50 secreting anti-IL8 antibodies described herein, the shaped particle pore size is small enough to prevent free diffusion of the target secretion, anti-IL-8. This causes secreted anti-IL-8 to bind to the surfaces of the shaped particle 10 (i.e., within the cavity as well as on the periphery of the outer edge), and strengthens the visibility of the accumulated fluorescent signal (FIGS. 9D-9E, 25A-25B). The shaped particles 10 and associated cells 50 are incubated in a cell incubator until sufficient secretions are produced and captured on the shaped particle surface (FIGS. 9D-9E, 25E).

(4) Staining the Shaped Particles with Attached Cells for Captured Biomolecules Using a Second Affinity Capture Agent Specific to the Secretion.

Once shaped particles 10 have been incubated long enough for sufficient secretions to have accumulated and bind on the shaped particles 10 for visualization, the shaped particles 10 can then be washed and labeled for visualization or separation. Several different reporting schemes can be used to analyze the secreted molecules bound to shaped particles 10. In one preferred embodiment a secondary antibody conjugated to a fluorophore which is specific against a second epitope on the secreted molecule can be added to form a fluorescent sandwich immunocomplex, reporting the presence of the bound secreted molecule. This method enables quantification through many commonly used analytical tools such as flow cytometers (illustrated in FIG. 23), plate readers, and fluorescence microscopes (FIGS. 25B-25C).

For secreted molecules present in particularly low concentrations, amplification schemes wherein reporter antibodies are conjugated to enzymes such as horseradish peroxidase that cleave fluorescent dyes that then can bind to shaped particles 10 can amplify signal, as described herein, such as through the use of tyramide chemistry. In a related embodiment, magnetic nanoparticles or magnetic particles can be used to label captured secreted molecules of interest. The addition of magnetic properties can be used in numerous ways. For example, to enrich shaped particles 10 of interest or to sort samples of interest using magnetic forces (e.g., magnetic activated cell sorting techniques). It should be appreciated that labeling is not limited to these two modalities (e.g., fluorescence and magnetic) and can include a combination of multiple modalities. Other modalities could include colorimetric, phosphorescence, light scattering particles, plasmonic nanoparticles, oligonucleotide barcodes, metal isotope tags, among others known in the art.

(5) Analyzing (e.g., with a Flow Cytometer) the Labeled Shaped Particles with Attached Cells and Optionally Sorting Cells of Interest Attached to the Shaped Particles Based on a Threshold of Intensity Based on Labeling Corresponding to Secretion Amount/Affinity.

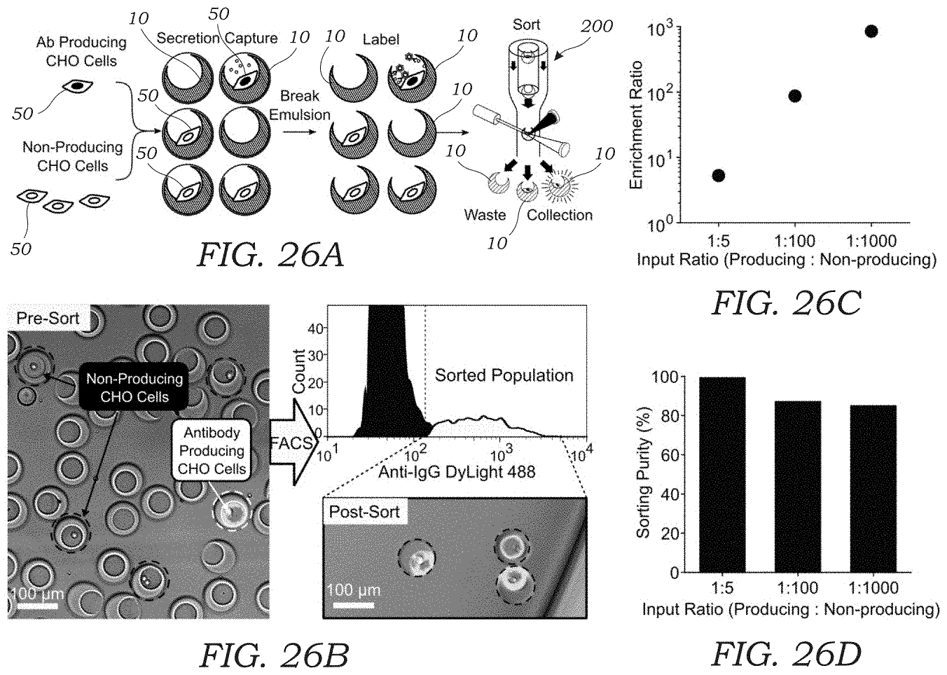

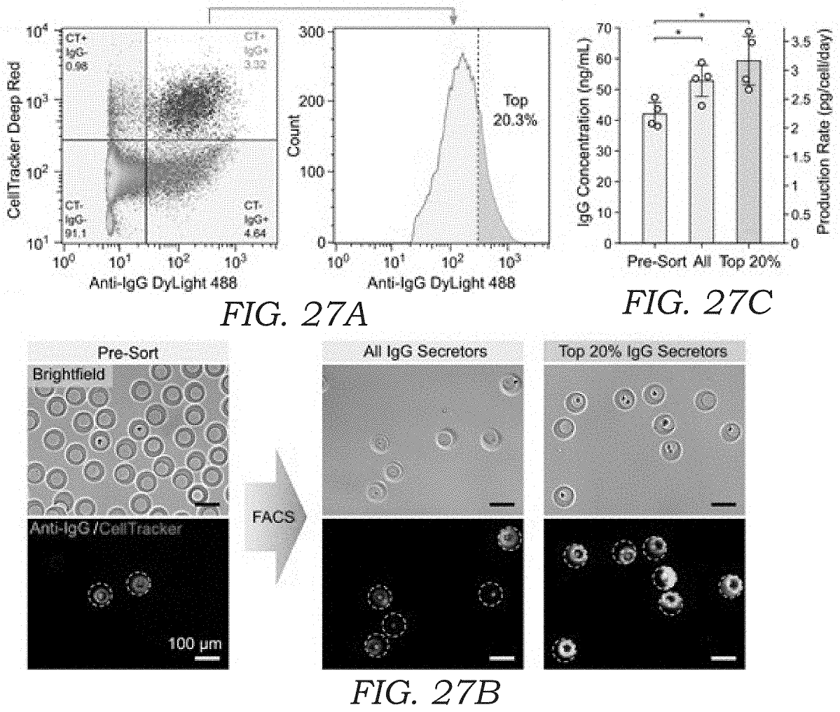

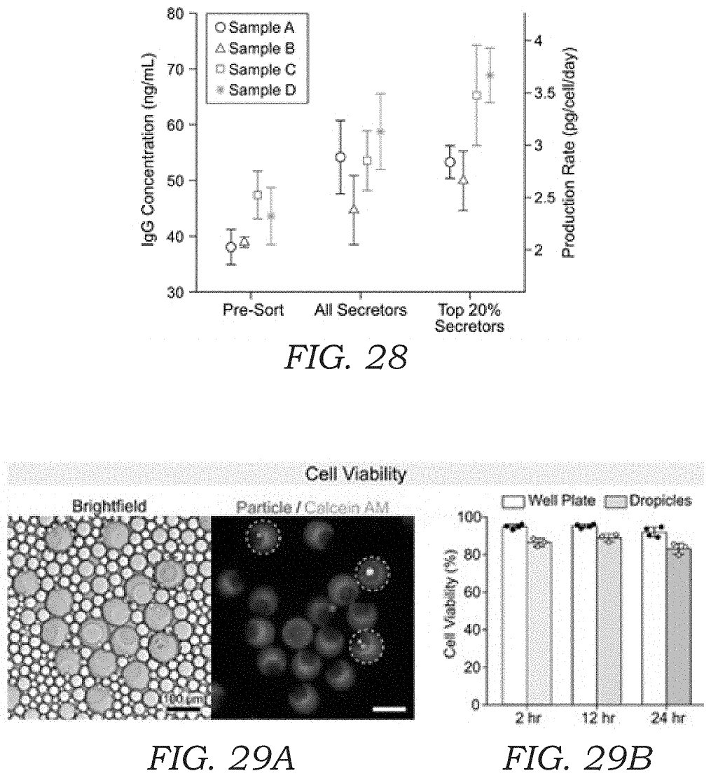

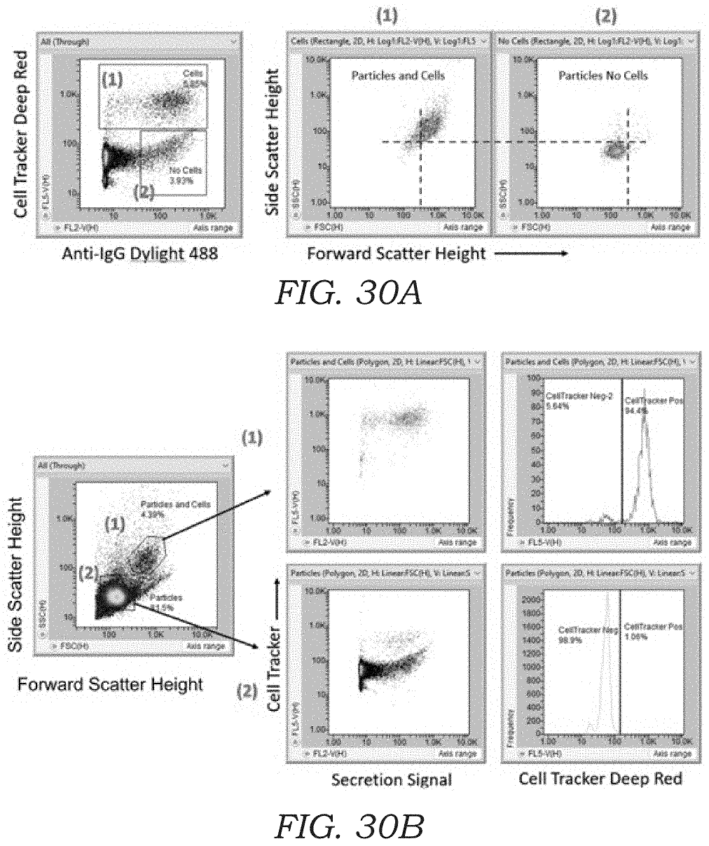

Once stained or otherwise tagged or modified to report a signal (e.g., light, magnetic, nucleic acid sequence), shaped particles 10 and their associated cells 50 can be analyzed, and also sorted, in high throughput using for example, commercially available flow sorters 200 (illustrated in FIGS. 9B, 23, 26A). In preferred embodiments, shaped particles 10 are suspended within nutrient enriched PBS such as PBS+2% FBS to preserve cell viability over the sorting process. The relative size of shaped particles 10 is tuned to enable clear distinction of particles 10 from contaminating dust, cell debris, or small diameter oil droplets in the solution during sorting allowing easy identification using forward scatter and side scatter signals. Adhered cells 50 stained with cytoplasmic tracking dyes, nuclear stains, viability stains, or reporter antibodies are also easily detected on shaped particles 10, enabling direct analysis of cell containing particles and gating of analysis or sorting to only analyze shaped particles 10 containing cells 50. For example, gating for intensity above a threshold for a cell-staining fluorescent dye will allow analysis of shaped particles 10 containing cells 50 only and exclude the analysis of empty shaped particles 10 exposed to secretions from cells 50 adhered in neighboring shaped particles 10 (FIGS. 27A and 30A). Alternatively, gating on scatter signal above a threshold level (e.g., side scatter height and forward scatter height or side scatter area and forward scatter height) can identify cell-containing shaped particles 10 for more accurate analysis without additional cell staining (FIG. 9C, FIGS. 30A, 30B). Lastly, reporter antibodies added to the surfaces of shaped particles 10 allow direct quantification of relative protein production from individual cell 50 clones within particle voids or cavities 12 from the relative fluorescence intensity of the shaped particle surface. Sorted cells 50 of interest, for example, clones secreting high levels of a desired protein are readily isolated, maintain a high secreting phenotype (FIGS. 26A-26D and 27A-27C), and remain viable for subsequent expansions, enabling enrichment of beneficial cell phenotypes (FIGS. 28 and 29A-29E). The void or cavity 12 of the shaped particle 10 in particular acts to prevent exposure to high shear stress to cells 50 adhered within the shaped particle 10 during flow through the fluid path of a flow cytometer or fluorescence activated cell sorter. For example, the shear stress exposure in the void or cavity 12 of the shaped particle 10 is reduced one order of magnitude from the shear stress on the outer surface of the shaped particle 10 as it flows, improving the viability of the cell 50 adhered in the cavity.

In other examples, screening of single cells 50 based on total secretion can be performed over multiple cycles to improve selection of desired subpopulations. For example, repeating the workflow depicted in FIG. 23. Following previously mentioned approaches, single cells 50 can be isolated into shaped particles 10 where secretions accumulate without crosstalk and are captured onto shaped particles 10. The shaped particles 10 can then be stained to indicate the quantity of secretions, and analyzed/sorted along with the attached cells 50. Sorted sub-populations of cells 50 can then be expanded to perform repeated selection steps. In one embodiment, sorted cells 50 attached to shaped particles 10 can be seeded into a well plate or flask and directly expanded from the shaped particles 10. For example, after several days of culture it was found that CHO cells 50 expanded across the surface of the shaped particle 10 eventually expanding on the surface of the well plate they were seeded in (FIGS. 29C-29E). This has the advantage of reducing processing steps and limiting trypsinization of cells 50. If desired, adherent cells 50 can be removed from the shaped particles 10 using standard trypsinization and passaging steps. After expansion of cells 50, the single cell secretion and sorting cycle can be performed again by seeding recovered cells 50 on shaped particles 10 again and repeating the previously outlined steps. In a related embodiment, single cells 50 seeded in the shaped particles 10 can be grown to create a clonal colony attached to a shaped particle 10 prior to analysis of released biomolecules. This enables the combined analysis/sorting based on growth and secretion of a clone. For example, cells 50 can be seeded into shaped particles 10 at a concentration such that most contain only a single cell 50. If desired, an initial population can be screened to remove shaped particles 10 with multiple cells (e.g., using a flow-sorter). After seeding cells 50 can be expanded directly on the shaped particles 10 such that a single cell colony is formed. This can be done over various times (e.g., <24 hours, 24 hours-1 week, >1 week) depending on the application. After colony formation, background signal can be washed away and shaped particles 10 can be modified with secretion binding sites. A secretion screen is then performed as previously outlined for single cells 50.

Example Secretion Workflow Using Shaped Particles—CHO Cells

An example workflow for selecting out high secreting CHO cells 50 is detailed as follows. The example cell-line used is CHO-DP12 clone #1934 (ATCC). Cell media was prepared as specified by ATCC. The CHO-DP12 cell line produces human anti-IL-8 antibodies which is the targeted secretion for this example experiment.

(1) Loading Cells to Attach to the Shaped Particles in an Aqueous Solution.

In this example, shaped particles 10 with an outer diameter of 82.5 microns, inner diameter of 50 microns were used. Shaped particles 10 were modified with 0.5 mg/ml of biotin-PEG-thiol (5000 MW, nanocs) and 4 mg/ml of RGD (added to the precursor materials during fabrication as previously described). 30 μL of concentrated shaped particles 10 were diluted with 1 mL of cell media and added into one well of a 12 well plate. Shaped particles 10 were then allowed to settle for 10 min. CHO DP-12 cells 50 were concentrated to 4 million cells per ml. For a target encapsulation of ˜0.3 cells per particle 18 μL of concentrated cell stock was taken and diluted to 50 μL with media, and then carefully transferred into the well pre-seeded with shaped particles. For a target encapsulation of ˜0.1 cells per particle, 6 μL of concentrated cell stock was diluted to 50 μL and then carefully transferred into the well pre-seeded with shaped particles 10 added. Cells 50 were allowed to seed for 10 min before moving the well plate into an incubator. It was found that a range of 4-12 hours was needed for cells 50 to attach strongly to the shaped particles 10.

(2) Washing Away Unbound Cells and/or Background Secretions and Adding an Affinity Capture Agent to Bind to the Shaped Particles that Captures a Specific Cell Secretion of Interest.

After cells 50 were incubated for a sufficient amount of time to attach to the shaped particles 10, shaped particles 10 were transferred from the well plate to a 15 mL conical tube. This was done by tilting the well plate at approximately a 15-30° angle and pipetting excess media from the top down to shear off shaped particles 10 sticking to the surface, and pipetting the dislodged shaped particles 10 and associated cells 50 to the 15 ml conical tube. To limit adhesion of shaped particles 10 to the walls of the Eppendorf conical tubes, the Eppendorf tubes can be pretreated with a solution of PBS with 0.1% Pluronic F-127, PBS with 0.5% BSA (Bovine serum albumin), PBS with 2% FBS (fetal bovine serum), or any combination of the above. Shaped particles 10 and associated cells 50 were then washed 2-3 times with a washing buffer consisting of PBS (with calcium and magnesium ions) supplemented with 0.5% BSA, 0.05% Pluronic F-127, and 1× Penicillin-Streptomycin. This wash removes any biotin that might be present in the media and any proteins from the cells 50 that may be present in the background media. Note for all washing steps samples were centrifuged at 300 g for 3 min. Supernatant is then removed by aspirating or pipetting leaving only the shaped particles 10 and cells 50 at the bottom of the conical tube. Particles 10 and cells 50 are then resuspended in 1-2 mL of the washing buffer and washing is repeated as necessary. After the final washing step, the pelleted particles and associated cells are modified by adding ˜60 μL of a 0.02 mg/ml working solution of streptavidin which binds to available biotin groups on the shaped particles. After incubating for 10 min., shaped particles 10 and associated cells 50 were washed 2-3 times with washing buffer. Next, the shaped particles 10 were modified with biotinylated IgG FC Goat anti-Human which is used as an example capture site for the secreted anti-IL-8 proteins. In this example, 60 μL of a working solution of biotinylated IgG FC Goat anti-Human (0.075 mg/mL) was added to each sample and then incubated for 10 min. Alternatively, biotinylated IgG1, FC Mouse Anti-Human, or biotinylated Protein A can be used. Finally, the samples were washed 2-3 times with PBS+0.5% BSA. On the final wash the PBS was replaced with cell culture media.

(3) Incubating the Shaped Particles for a Time Period to Accumulate Secretions that Bind with the Affinity Capture Agent.

Samples were allowed to incubate for 2-24 hours in a cell incubator (37° C., 5% CO₂). During this step secreted Anti-IL-8 proteins attach to Anti Human IgG Fc binding sites on the shaped particles (see FIG. 25A).

(4) Staining the Shaped Particles with Attached Cells for Captured Secretions Using a Second Affinity Capture Agent Specific to the Secretion.

Shaped particle 10 samples in aqueous phase are washed 2-3 times with a sorting solution composed of PBS (with calcium and magnesium ions), 2% FBS, 0.05% Pluronic F-127, and 1× Penicillin-Streptomycin. On the last wash samples are spun down and supernatant is aspirated until ˜60 μL remains. 60 μL of a fluorescent secondary working solution (0.033 mg/mL Goat Anti-Human IgG H&L (Dylight 488) preabsorbed (ab96911) was added to the sample to stain captured Anti-IL-8 proteins captured on the shaped particles (FIG. 25A). After incubating for 30 min at 37° C. the samples were then washed 2-3× with PBS+2% FBS to remove any un-conjugated stain. Samples were then put on ice during transfer to the flow cytometer.

Optionally, during this staining process, cells 50 associated with the shaped particles 10 can be stained with Calcein AM or CellTracker dye. FIG. 29A shows example microscope images taken of shaped particles 10 with an associated cell stained with Calcein AM. FIGS. 25B, 25F, 25G and FIG. 26B show example microscopy images of cells 50 with Cell Tracker dye and secretion labels on associated shaped particles 10. Fluorescence imaging clearly shows a strong signal in the Cy5 channel (secretion stain channel) on the shaped particle 10 with the CellTracker-stained cells 50 present.

(5) Analyzing (e.g., with a Flow Cytometer) the Stained Shaped Particles with Attached Cells and Optionally Sorting Cells of Interest Attached to the Shaped Particles Based on a Threshold of Intensity Based on Staining Corresponding to Secretion Amount/Affinity.

Samples were analyzed in high-throughput using the On-Chip Sort flow cytometer from On-Chip Biotechnologies Co., Ltd., Tokyo, Japan. Samples were diluted to 200 μL with PBS+2% FBS and added to the sample inlet. Here, the 150 μm flow chip from On-Chip biotechnologies was used. PBS+2% FBS and 1× Penicillin-Streptomycin was also used as the sheath fluid. Gating on the forward scatter height (FSC (H)) and side scatter height (SSC(H)) was used to select out shaped particles from other background events/noise (see e.g., FIG. 30B). Other events may be associated with cells 50 dissociated from shaped particles 10, or cell debris. Example gating plots are shown in FIGS. 27A and 30A. After this first gating, sorting events were then selected from relative far-red (deep red) fluorescence signal. For example, in FIG. 27A the top 10% of far-red signal was selected as a sorting gate. Samples containing shaped particles 10 with attached cells 50 were then sorted and collected. Using the On-Chip Sorter flow cytometer, event rates were typically around 50-300 events/s for the samples. Collected samples were stained with Calcein AM and visualized under fluorescence microscopy as shown FIG. 27B. After the sort, a large fraction of the cells 50 maintained viability as shown by the Calcein AM live stain signal. Further images of the Cy5 channel (captured secretion stain channel) show clear accumulation of shaped particles 10 and associated cells 50 with high levels of captured secretions after the sort (e.g., top 20% IgG secretors with reference to FIGS. 27B-27C and 28). This demonstrates the ability to sort cells 50 based off of relative secretion levels using the shaped particle system.

Dropicles

In related embodiments to those described above, an optional dropicle formation step can be included between step (2) washing, and step (3) incubating. In addition, a dropicle emulsion-breaking step can be included after the (3) incubating step. In the dropicle formation step, shaped particles 10 with loaded cells 50 are mixed with an oil phase including surfactant to form monodisperse droplets around the shaped particles 10 (FIGS. 24D-24F). The oil phase prevents the transport of secreted molecules or other biomolecules released from cells 50 and can increase the amount captured on the shaped particle affinity capture sites. The void or cavity 12 of the shaped particles 12 protects the cells 50 from shearing and damage from the oil-water interface around the exterior surface of the shaped particles 10. Dropicles can be formed with generally monodispersed sizes and loaded with a single shaped particle 10 per dropicle (FIGS. 24E and 24F).

An example workflow to form dropicles with shaped particles 10 is as follows: Shaped particles 10 were suspended in cell culture media and then concentrated by centrifuging at 300 g for 3 minutes and aspirating supernatant. An oil phase made with Novec™ 7500 and 2% w/w PicoSurf™ was added to the particle suspension at approximately 2× the remaining volume. The sample was then vigorously pipetted for 30 s (˜50 pipettes) using a 200 μL micropipette (Eppendorf) to generate dropicles (FIG. 24D, 29A).

Following incubation in dropicles shaped particles 10 and associated cells 50 can be recovered back into an aqueous phase for further analysis. To recover cells 50 back into an aqueous phase excess oil was first removed via pipetting and several ml of media was added on top of the emulsions. To destabilize the droplets 50 μL of 20% v/v perfluorooctonal (PFO) in Novec™ 7500 was then pipetted on top of the emulsion layer and the sample was gently agitated. After 5 min most of the droplets were merged and particles 10 and associated cells 50 transferred into the bulk media phase. Viability of the cells is maintained through this process. See, e.g., FIG. 29B. Optionally, samples can be centrifuged for 15-30 s at 200 g to coalesce remaining droplets. The suspension of particles 10 and cells 50 were then transferred by pipetting into a separate conical tube. Cell populations may be expanded post dropicle release as seen in FIGS. 29C and 29D.

Use of shaped particles as scaffolds for cell and tissue growth. A plurality of shaped particles 10 with voids or cavities 12 as described in multiple embodiments herein that also have cell-adhesive groups (e.g., RGD) and optionally degradable crosslinker components (e.g., MMP-degradable peptide crosslinkers) can be used as a scaffold to promote cell growth in vitro or cell and tissue growth in vivo after injection or implantation. The higher void fraction of the shaped particles 10 compared to spherical particles can lead to increased cellular ingrowth and cellularity prior to material degradation while maintaining structural integrity of the scaffold. The higher void fraction can also enhance diffusive and convective transport through the scaffold to increase the viability and tissue penetration depth within the scaffold. Preferably, the void or cavity 12 in the shaped particles 10 is configured to not geometrically nest, which would lead to reduced void fraction in a plurality of nested particles 10. For example, for a crescent shaped particle 10, the outer radius of curvature of the envelope of the shaped particle 10 should be greater than the radius of curvature of the cavity within the shaped particle 10.

A plurality of shaped particles 10 can be injected as a slurry into a tissue to treat disease in the tissue, to build additional tissue, or to encourage tissue growth to heal a wound. In some embodiments, cells 50 adhered to a plurality of shaped particles 10 can be injected as a slurry into a tissue to treat disease in the tissue, to build additional tissue, or to encourage tissue growth to heal a wound. Cells 50 may include stem cells, such as mesenchymal stem cells, neural stem cells or the like. In a preferred embodiment one of the precursor materials of the shaped particles include a PEG phase having RGD peptide, K peptide, and Q peptide and further includes an MMP-degradable peptide linker as described in (U.S. Patent Application Publication No. 2017/0368224) for spherical particles, which is incorporated herein by reference. As described in U.S. Patent Application Publication No. 2017/0368224, in some embodiments the shaped particles 10 can be injected into a tissue or wound site and annealed following injection using an annealing agent (e.g., Factor XIIIa, Eosin Y and light, or other chemistries used for annealing as described therein). Following annealing the individual shaped particles 10 are linked to the surface of adjacent contacting particles 10 and adjacent contacting tissue to form a contiguous stable scaffold material.

In related embodiments, shaped particles 10 with a void or cavity 12 that is completely surrounded by hydrogel material (e.g., FIG. 1B), also called a capsule particle 10, is used in hydrogel scaffolds. In this embodiment cells 50 cannot immediately migrate into or grow within the void or cavity 12 of the capsule particle 10 but can migrate around and in the interstitial space between particles 10. However, at a later time point as the outer hydrogel material degrades the void or cavity space 12 of the capsule becomes accessible for cell migration and growth. This capsule particle 10 therefore, can provide advantages to allow increased space for tissue growth following a delayed time period required for material degradation. In related embodiments high molecular weight agents, such as drugs, antibodies, antigens, adjuvants or other biologicals or small molecules drugs attached to high molecular weight polymers, can be loaded during manufacture in the precursor solution such that it is maintained in the void or cavity space 12 of the capsule following crosslinking. Upon degradation of the capsule wall a burst release of the loaded agent is triggered, which can be advantageous to deliver medications, growth factors, antigens/adjuvants for vaccines, into a tissue in a time-delayed manner, but still at high concentration, which is difficult with other current drug delivery techniques.

Device Fabrication—Step Emulsification Device

In one embodiment a microfluidic step emulsification device 20 (e.g., FIG. 13) can be used to generate uniform droplets in high-throughput. For device fabrication, master molds are fabricated on silicon wafers using a two-layer photolithography process. The first and second layers define the nozzle channel 26 height and the inlet/outlet reservoir region channel 32 height, respectively. For example, a device 20 with nozzle channel 26 height of 25 microns and reservoir channel 32 height of 150 microns would be fabricated as follows. Spin coat KMPR1025 at a rate of 3200 RPM for 30 seconds and soft bake for 12 minutes at 100° C. Expose the first layer using either a chrome or transparency mask on a mask aligner (e.g., Karl Suss MA6). Expose for 24 seconds at 12 mW and follow with a 2-minute post exposure bake at 100° C. Spin coat KMPR1050 directly on the first layer at 1000 RPM for 30 seconds and then soft bake for 30 minutes at 100° C. Using the mask aligner align the second mask layer to the first layer using alignment marks and then expose for 83 seconds at 12 mW. Then do a 5-minute post exposure bake at 100° C. Remove any unexposed photoresist by developing with SU8 Developer for approximately 10 minutes followed by ˜1 minute Isopropanol wash and then dry with Nitrogen gas. Molds with different channel heights can be fabricated by adjusting the photoresist types (e.g., KMPR 1005, KMPR 1010) and the spins speeds following general parameters given in the photoresist data sheets.

PDMS tops 24 can be molded from the master molds using poly(dimethyl)siloxane (PDMS) Sylgard 184 kit (Dow Corning). In one example, the base and crosslinker were mixed at a 10:1 mass ratio, poured over the mold, degassed, and cured at 65° C. overnight. The PDMS top 24 and glass substrate 22 (e.g., microscope slides (VWR)) were then activated via air plasma for 30-90 seconds (Plasma Cleaner, Harrick Plasma) and bonded together by placing in contact. The devices 22, 24 are then placed back in the oven for at least 30 minutes. For use with fluorinated oils the channel surfaces are modified to be fluorophilic to achieve proper droplet formation. In one approach the bonded devices were treated with Aquapel™ for 1 min by injecting with a glass syringe and then rinsed with filtered Novec™ 7500 oil (3M). In another approach a solution of Novec™ 7500 oil+1-5% Trichloro(1H,1H,2H,2H-perfluorooctyl)silane is injected into the devices 20 using a syringe. The solution is left to sit for 1-5 minutes and then removed with air and flushed with filtered Novec™ 7500 oil (3M). In both approaches the devices 20 are placed back in the oven at 70° C. for at least 30 min to evaporate residual oil in the channels.

Phase separation of a solution of PEG and gelatin by changing temperature: Here experiments are described that identify the concentration of PEG and gelatin precursor solutions that remain mixed in an aqueous solution at room temperature but that can split into two phases upon cooling. Two samples of solutions were made for each condition and incubated at room temperature and 4° C. respectively. After 4 hours of incubation, phase separation was checked and plotted in a graph for a better visualization (FIGS. 4A and 5). The binodal for each temperature was estimated based on the results. Different molecular weight PEG precursors, including 700, 1500, 10000 and 20000 Da, were used to consider the effect PEG molecular weight had on the temperature induced phase separation behavior. Since a higher molecular weight of PEG tends to start phase-separation at a lower total polymer concentration, we decreased the concentration of both PEG and gelatin precursors for the test of high molecular weight PEG.

Fabrication of crescent shaped particles using temperature induced phase separation: In order to fabricate crescent shaped particles 10 using the induced phase separation approach, first, droplets were fabricated using a step-emulsification multi-channel high-throughput PDMS microfluidic droplet generator device 20. A solution of 7.5% w/w PEGDA 1500 Da (Sigma), 15% w/w cold water fish gelatin (Sigma) and 1% w/w LAP (Lithium phenyl-2,4,6-trimethylbenzoylphosphinate, Sigma) in Phosphate Buffered Saline (PBS, pH7.2) was prepared. Next, the solution was injected into a PDMS microfluidic device 20 at a rate of 10 μL/min using a syringe pump (Harvard Apparatus PHD 2000). An oil phase made with Novec™ 7500 (3M) and 0.25% w/w PicoSurf (Sphere Fluidics) was injected at a rate of 42 μL/min to partition the aqueous phases into monodisperse water and oil droplets. The microfluidic device 20 was operated at room temperature to maintain a mixed state for the precursor materials. Droplets were collected into a PDMS reservoir channel 32 and incubated in a 4° C. refrigerator for 1 hour to allow phase separation of PEG and gelatin polymers (FIG. 14A). After phase separation, the PEG phase was crosslinked with UV light at a power of 10-30 mW/cm² over an approximate duration of 5 minutes. Crosslinked shaped particles 10 were collected and oil and un-crosslinked gelatin were removed using a series of washing steps. Briefly, excess oil was removed by pipetting and a layer of PBS was added on top of the remaining emulsions. A solution of 20% v/v perfluorooctanol (PFO, Sigma) in Novec™ 7500 was then added to destabilize the emulsions and transfer particles to the PBS phase. Excess oil was removed and samples were washed 2× with Novec™ 7500 to remove remaining surfactant. Novec™ 7500 was removed by pipetting and residual oil was removed by washing 2-3× with hexane (Sigma). Samples were then washed 3× with PBS to remove gelatin from the system.

While embodiments of the present invention have been shown and described, various modifications may be made without departing from the scope of the present invention. The invention, therefore, should not be limited, except to the following claims, and their equivalents.

1-24. (canceled)

25. A shaped particle system comprising:
a plurality of shaped particles having a diameter of <70 μm, each shaped particle having a void or cavity formed therein, wherein the shaped particles are three-dimensional and comprise one of: crescent shaped, bowl shaped, or moon shaped, and wherein each shaped particle comprises a poly(ethylene glycol) (PEG) component located in a first region of the shaped particle and a cell adhesive layer located in a second region of the shaped particle.

26. The shaped particle system of claim 25, wherein the cell adhesive layer comprises a localized gelatin region located along a surface of the void or cavity.

27. The shaped particle system of claim 25, wherein the cell adhesive layer comprises one or more cell adhesive moieties located along a surface of the void or cavity.

28. The shaped particle system of claim 26, wherein the localized gelatin region comprises one or more affinity capture agents.

29. The shaped particle system of claim 25, further comprising a cell adhered to the shaped particle within the void or cavity.

30. The shaped particle system of claim 29, wherein the plurality of shaped particles comprise an emulsion wherein an aqueous phase fluid is disposed in the void or cavity and the plurality of shaped particles are carried by an oil-based fluid.

31. The shaped particle system of claim 25, wherein the diameter of an opening of the void or cavity is smaller than a diameter of the void or cavity.

32. The shaped particle system of claim 29, wherein a ratio of a characteristic dimension of the cell to the void or cavity is within the range of about 0.5 to about 1.

33. A method of performing a cell secretion assay using shaped particles comprising:
a. providing a plurality of shaped particles having a diameter of <70 μm, each shaped particle having a void or cavity formed therein;
b. loading cells into the voids or cavities of the plurality of shaped particles;
c. adding an affinity capture agent to the plurality of shaped particles specific to a cell secretion of interest;
d. incubating the plurality of shaped particles with the loaded cells;
e. adding a stain, dye, label, or other secondary affinity capture agent specific to the secretion of interest on or in one or more of the plurality of shaped particles; and
f. analyzing or sorting the plurality of shaped particles of operation (e) based on a signal formed or property generated by the stain, dye, label, or other secondary affinity capture agent specific to the cell secretion of interest on or in one or more of the plurality of shaped particles.

34. The method of claim 33, wherein analyzing or sorting the plurality of shaped particles of operation (f) comprises flowing the plurality of shaped particles through a flow cytometer, fluorescence activated cell sorter, or other single-cell analysis instrument.

35. The method of claim 34, further comprising sorting the plurality of shaped particles based on a threshold or gate in fluorescence intensity, scatter intensity, or other signal measured with the flow cytometer, fluorescence activated cell sorter, or other single-cell analysis instrument.

36. The method of claim 33, wherein the shaped particles are three-dimensional and comprise one of: crescent shaped, bowl shaped, or moon shaped.

37. The method of claim 33, wherein operation (c) is performed prior to operation (b).

38. The method of claim 33, wherein the analyzing or sorting operation comprises analyzing or sorting >10,000 shaped particles.

39. The method of claim 35, further comprising:
g. culturing the cells loaded on the shaped particles following sorting the plurality of shaped particles.

40. The method of claim 33, wherein the diameter of an opening of the void or cavity is smaller than a diameter of the void or cavity.

41. The method of claim 33, wherein a ratio of a characteristic dimension of the cell to the void or cavity is within the range of about 0.5 to about 1.

42. The method of claim 36, wherein the shaped particles comprises a cell adhesive layer comprising a localized gelatin region located along a surface of the void or cavity

43. A method of analyzing or sorting cells adhered to shaped particles with a flow cytometer comprising:
a. providing a plurality of shaped particles having a diameter of <70 μm, each shaped particle having a void or cavity formed therein, wherein the shaped particles are three-dimensional and comprise one of: crescent shaped, bowl shaped, or moon shaped;
b. loading cells into the voids or cavities of the plurality of shaped particles;
c. flowing the plurality of shaped particles through a flow cytometer; and
d. analyzing the plurality of shaped particles of operation (c) based on a fluorescence and/or scatter signal measured with the flow cytometer.


SEQUENCE LISTING (1 sequence)

SEQ ID NO: 1 - protein, 10 aa, Artificial Sequence
  Cell adhesive peptide
RGDSPGERCG